(19) United States
(12) Patent Application Publication
(10) Pub. No.: US 2024/0025908 A1
(43) Pub. Date: Jan. 25, 2024

(54) COMPOUND USED AS KINASE INHIBITOR AND USE THEREOF

(71) Applicant: TYK MEDICINES, INC., Huzhou, Zhejiang (CN)

(72) Inventors: Jun LI, Huzhou, Zhejiang (CN); Apeng LIANG, Huzhou, Zhejiang (CN); Chengshan NIU, Huzhou, Zhejiang (CN); Yusheng WU, Huzhou, Zhejiang (CN)

(21) Appl. No.: 18/251,729

(22) PCT Filed: Nov. 3, 2021
(86) PCT No.: PCT/CN2021/128501

§ 371 Date: May 4, 2023

(30) Foreign Application Priority Data
Nov. 4, 2020 (CN) 202011216420.5

(51) Int. Cl. C07D 487/04; A61P 35/00

(52) U.S. Cl. CPC C07D 487/04; A61P 35/00

(57) ABSTRACT

A compound represented by formula (I) or a stereoisomer, a tautomer, a crystal form, a pharmaceutically acceptable salt, a hydrate, a solvate or a prodrug thereof are provided. The compound represented by formula (I) can be used as a kinase inhibitor for preparing a medicament for treating diseases mediated by kinase such as ROS1, NTRK and ALK.

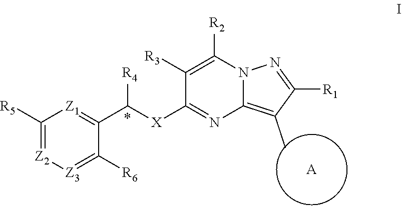

### TECHNICAL FIELD

The invention relates to the technical field of medicine, in particular to a compound used as a kinase inhibitor, a preparation method thereof, and use for preparing a medicament for treating diseases mediated by kinase such as ROS1, NTRK, ALK.

### BACKGROUND OF THE INVENTION

ROS1 (c-ros oncogene 1 receptor kinase) is a tyrosine protein kinase encoded by ROS1 proto-oncogene in human body. It is located on chromosome 6q22.1 and belongs to the tyrosine kinase insulin receptor gene. It is composed of intracellular tyrosine kinase active region, transmembrane region and extracellular region, and encodes chimeric protein with tyrosine kinase activity. The basic structure consists of extracellular N-terminal ligand binding region (amino acid 1-1861), transmembrane region (amino acid 1862-1882) and intracellular C-terminal tyrosine kinase active region (amino acid 1883-2347) consisting of 464 amino acids. When ROS1 gene rearranges, the extracellular region is lost, and the transmembrane region and intracellular tyrosine kinase region are retained. The rearrangement sites mainly occur in exons 32-36 of ROS1 gene. ROS1 gene mutation mainly occurs in lung cancer patients, and the proportion of patients is 1%-2%. In NSCLC, ROS1 gene mainly fuses with SLC34A2 and CD74, and continuously activates ROS1 tyrosine kinase region and downstream signaling pathways such as JAK/STAT, PI3K/AKT, RAS/MAPK, thus causing tumor occurrence. It has been proved in a large number of literatures and clinically that diseases caused by ROS1 overactivation, especially cancer, can be treated by inhibiting the activity of mutated ROS1 kinase. At present, crizotinib and entrectinib are on the market for the treatment of ROS1 positive non-small cell lung cancer, both of which belong to the first generation of small molecule ROS1 inhibitors. However, during the treatment of crizotinib or entrectinib, drug resistance and disease progression will occur in about 15 months. Among drug-resistant patients, the most common drug-resistant mutation is solvent front mutation such as G2032R. For drug-resistant patients, there are no therapeutic drugs on the market at present. Therefore, it is urgent to develop new inhibitors against ROS1, especially new ROS1 inhibitors against drug resistance to the first generation of ROS1 inhibitors such as crizotinib or entrectinib for clinical treatment.

Tropomyosin receptor kinase (TRK) family belongs to transmembrane receptor tyrosine kinases (RTKs), which are involved in regulating synaptic growth and function maintenance, memory generation and development, and protecting neurons from damage, etc. in mammalian nervous system. TRK kinase is a kind of nerve growth factor receptor. Its family consists of Tropomyosin-related kinase A (TRKA), Tropomyosin-related kinase B (TRKB) and Tropomyosin-related kinase C (TRKC), which are highly homologous and encoded by NTRK 1, NTRK 2 and NTRK 3 genes, respectively. Complete TRK kinase consists of extracellular domain, transmembrane domain and intracellular domain. Like other RTKs, the extracellular domain of TRK kinase binds with corresponding ligands to form dimer, which can cause autophosphorylation of intracellular domain of TRK kinase to activate its kinase activity and further activate downstream signal transduction pathway. TRK kinase affects cell proliferation, differentiation, metabolism and apoptosis through downstream pathways such as Ras/MAPK, PI3K/AKT and PLC 7. When the NTRKs gene is fused or mutated, the extracellular receptor is altered or eliminated (Greco, A. et. al, Mol. Cell. Biol. 1995, 15, 6118; Oncogene 1998, 16, 809), but the fused or mutated TRK protein is in a highly activated kinase activity state without ligand binding, which can continuously activate the downstream signal transduction pathway, causing the regulation disorder of the downstream signal pathway of TRK kinase, inducing cell proliferation and promoting the occurrence and development of tumor. NTRKs gene fusion occurs in a variety of solid tumors in adults and children, including breast cancer, colorectal cancer, non-small cell lung cancer, papillary thyroid cancer, Spitz-like melanoma, glioma and various sarcomas, etc. In common cancers, such as non-small cell lung cancer and colorectal cancer, the incidence of NTRK gene fusion is lower, about 1%-3%, but in some rare cancers, such as infantile fibrosarcoma and secretory breast cancer, the incidence of NTRK gene fusion can reach more than 90%. The earliest TPM3-TRKA fusion protein was found in colon cancer cells. Later, more types of NTRK fusion proteins such as CD74-NTRKA, MPRIP-NTEKA, QKI-NTRKB, ETV6-NTRKC, BTB1-NTRKC were found in different clinical tumor patients, such as breast cancer, non-small cell lung cancer, papillary thyroid cancer, Spitz-like melanoma, glioma. Therefore, in recent years, NTRK fusion protein has become an effective anti-cancer target, and has become a hot spot in the research and development of anti-cancer drugs. With the further understanding of TRK kinase in recent years, more TRK fusion protein types and mutation types have been found (Russo, M. et. al Cancer Discovery, 2016, 6, 36; Drilon, A. et. al, Annals of Oncology, 2016, 27, 920), so it is urgent to develop new NTRK inhibitors with better activity and wider effects in clinic, so as to solve the tumor treatment problems caused by these NTRK protein fusion or mutation.

2-5% of NSCLC patients are anaplastic lymphoma kinase (ALK) rearrangements, a receptor protein tyrosine phosphokinase in the insulin receptor superfamily. At first, people found ALK in the form of an activated fusion oncogene in anaplastic large cell lymphoma, and then continuous studies found the fusion form of ALK in various cancers, including systemic dysplasia, inflammatory myofibroblastic carcinoma, non-small cell lung cancer, etc. The mutation and abnormal activity of ALK in a variety of cancers have made it a drug target for the treatment of ALK-positive cancers. At present, there are many ALK kinase inhibitors on the market. With the clinical application of these drugs, patients will have drug resistance mutations. If G1202R and other drug resistance mutations occur, these drugs will lose their efficacy.

In recent years, with the further understanding of ROS1, NTRK, ALK and other kinases, and the increase of clinical drug-resistant patients, it is urgent to develop new tyrosine kinase inhibitors with better activity and wider effects in clinic, so as to solve the treatment problems of tumors caused by the fusion or mutation of ROS1, NTRK, ALK and other kinases.

### SUMMARY OF THE INVENTION

In the first aspect of the present invention, it provides a compound of formula (I), or a stereoisomer, a tautomer, a crystalline form, a pharmaceutically acceptable salt, a hydrate, a solvate or a prodrug thereof:

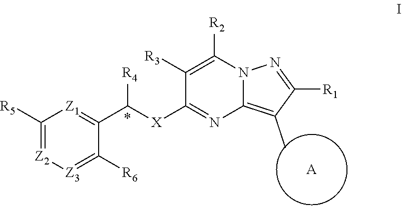

wherein,

* is R or S configuration;

Z₁, Z₂ and Z₃ are each independently selected from the group consisting of N and CR₁₃;

X is selected from the group consisting of NR₆, O, CR₁R₂, S, S(O) and S(O)₂;

R₁, R₂, R₃, R₄, R₅, R₆ and R₁₃ are each independently selected from the substituted or unsubstituted group consisting of H, halogen, amino, cyano, nitro, hydroxyl, acyl, ester group, C1-C6 alkyl, C3-C8 cycloalkyl, 3-8 membered heterocyclyl, C1-C6 alkoxy, C6-C14 aryl and 5-14 membered heteroaryl; wherein the substituted means being substituted by one or more R;

A is selected from the group consisting of

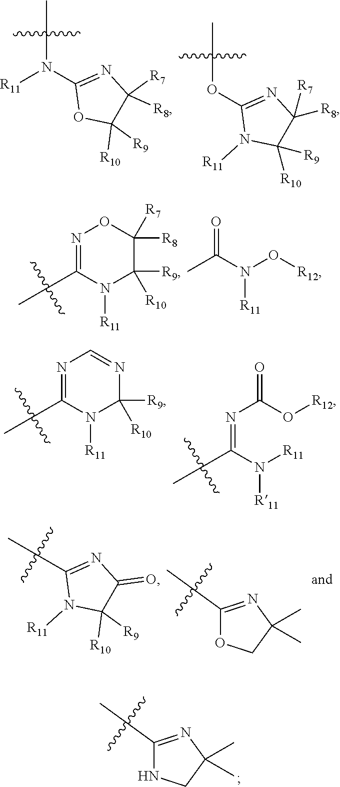

R₇, R₈, R₉, R₁₀, R₁₁ and R′₁₁ are each independently selected from the substituted or unsubstituted group consisting of hydrogen atom, cyano, C1-C6 alkyl, C3-C8 cycloalkyl, C6-C14 aryl and 5-14 membered heteroaryl; wherein the substituted means being substituted by one or more R;

R₁₂ is selected from the group consisting of C1-C6 alkyl and hydroxy-substituted C1-C6 alkyl;

R is selected from the group consisting of deuterium, halogen, amino, cyano, nitro, hydroxy, acyl, ester group, C1-C6 alkyl, C1-C6 haloalkyl, C3-C8 cycloalkyl, C3-C8 halocycloalkyl, C1-C6 alkoxy, C1-C6 haloalkoxy, C6-C14 aryl and 5-14 membered heteroaryl

In another preferred embodiment,

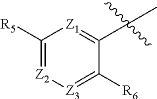

moiety is selected from the substituted or unsubstituted group consisting of phenyl and pyridyl;

wherein, the “substituted” refers to being substituted by one or more groups selected from the group consisting of halogen, cyano, nitro, hydroxyl, C1-C6 alkyl, C1-C6 haloalkyl, C1-C6 alkoxy and C1-C6 haloalkoxy.

In another preferred embodiment, the compound of formula I, or a stereoisomer, a tautomer, a crystalline form, a pharmaceutically acceptable salt, a hydrate, a solvate or a prodrug thereof has a structure shown in formula II:

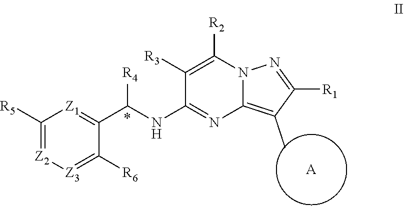

wherein,

* is R or S configuration;

Z₁, Z₂, Z₃, R₁, R₂, R₃, R₄, R₅, R₆ and A are as defined above.

In another preferred embodiment, the compound of formula I, or a stereoisomer, a tautomer, a crystalline form, a pharmaceutically acceptable salt, a hydrate, a solvate or a prodrug thereof has a structure shown in formula III or IV:

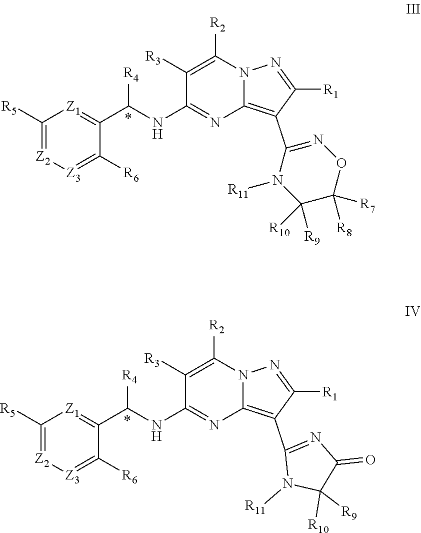

wherein,

* is R or S configuration;

Z₁, Z₂, Z₃, R₁, R₂, R₃, R₄, R₅, R₆, R₇, R₈, R₉, R₁₀ and R₁ are as defined above.

In another preferred embodiment, Z₁ is CR₁₃, preferably CH.

In another preferred embodiment, Z₂ is CR₁₃, preferably CH.

In another preferred embodiment, the compound of formula I, or a stereoisomer, a tautomer, a crystalline form, a pharmaceutically acceptable salt, a hydrate, a solvate or a prodrug thereof has a structure shown in formula V or VI:

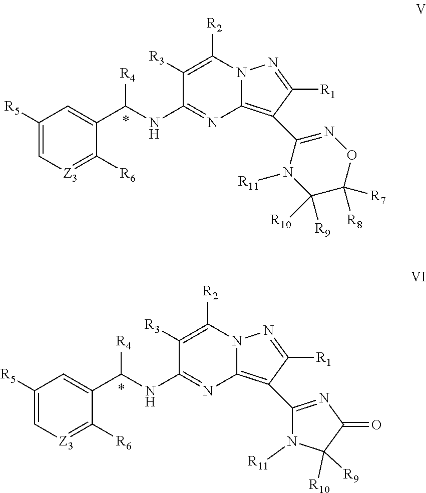

wherein,

* is R or S configuration;

R₄ is selected from: H, C1-C6 alkyl, C3-C6 cycloalkyl or C1-C6 haloalkyl;

Z₃, R₁, R₂, R₃, R₅, R₆, R₇, R₈, R₉, R₁₀ and Ru are as defined above.

In another preferred embodiment, the compound of formula I, a stereoisomer, a tautomer, a crystalline form, a pharmaceutically acceptable salt, a hydrate, a solvate or a prodrug thereof, wherein, R₁, R₂ and R₃ are each independently selected from the group consisting of hydrogen, halogen and amino;

R₄ is selected from the group consisting of hydrogen, C1-C6 alkyl, C3-C6 cycloalkyl, and C1-C6 haloalkyl;

R₅ is selected from the group consisting of hydrogen and halogen;

R₆ is selected from the group consisting of hydrogen, halogen, C1-C6 alkyl, C3-C6 cycloalkyl, C1-C6 alkoxy, C1-C6 alkylamino, C1-C6 haloalkyl, C1-C6 haloalkoxy, C1-C6 haloalkylamino and C3-C6 halocycloalkyl;

R₇, R₈, R₉, R₁₀ and Ru are each independently selected from the group consisting of hydrogen and substituted or unsubstituted C1-C6 alkyl; wherein, the “substituted” refers to being substituted by one or more groups selected from the group consisting of deuterium, halogen, amino, cyano, hydroxyl, acyl, ester group, C1-C6 alkyl, C1-C6 haloalkyl, C3-C6 cycloalkyl, C3-C6 halocycloalkyl, C1-C6 alkoxy, C1-C6 haloalkoxy, C6-C10 aryl and 5-10 membered heteroaryl.

In another preferred embodiment, the compound of formula I, or a stereoisomer, a tautomer, a crystalline form, a pharmaceutically acceptable salt, a hydrate, a solvate or a prodrug thereof has a structure shown in formula VII or VIII:

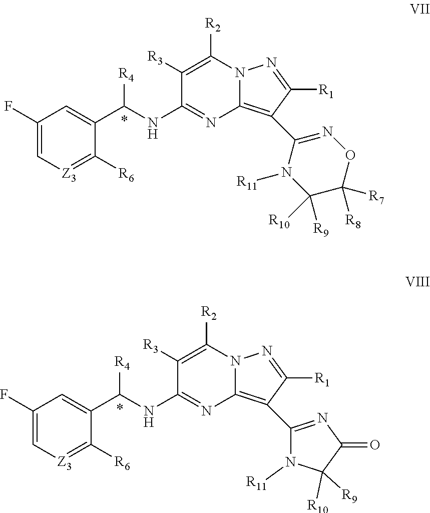

wherein,

* is R or S configuration;

Z₃, R₁, R₂, R₃, R₄, R₆, R₇, R₈, R₉, R₁₀ and Ru are as defined above.

In another preferred embodiment, R₆ is selected from the group consisting of halogen, C1-C3 haloalkoxy and C1-C6 haloalkylamino.

In another preferred embodiment, in formula I, R₅ is F.

In another preferred embodiment, R₇, R₈, R₉ and R₁₀ are each independently selected from the group consisting of hydrogen, C1-C3 alkyl and C1-C3 haloalkyl.

In another preferred embodiment, in formula I, Z₁, Z₂, Z₃, R₁, R₂, R₃, R₄, R₅, R₆, X and A are the groups corresponding to the specific compounds in the examples.

In another preferred embodiment, the compound of formula I, a stereoisomer, a tautomer, a crystalline form, a pharmaceutically acceptable salt, a hydrate, a solvate or a prodrug thereof, wherein, and the compound is selected from the group consisting of

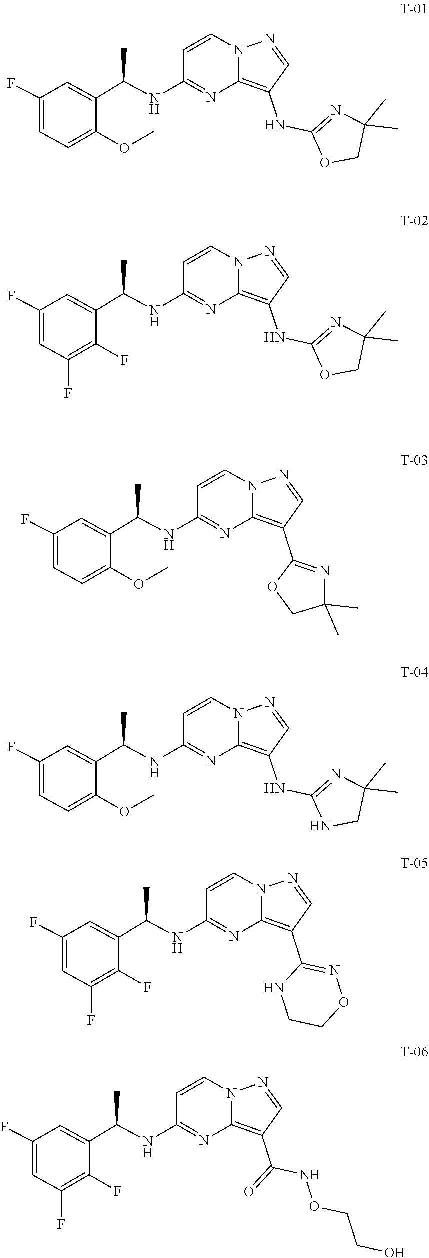 [SMILES: COc1ccc(F)cc1[C@@H](C)Nc1ccn2ncc(NC3=NC(C)(C)CO3)c2n1 ; C[C@@H](Nc1ccn2ncc(NC3=NC(C)(C)CO3)c2n1)c1cc(F)cc(F)c1F ; COc1ccc(F)cc1[C@@H](C)Nc1ccn2ncc(C3=NC(C)(C)CO3)c2n1 ; COc1ccc(F)cc1[C@@H](C)Nc1ccn2ncc(NC3=NC(C)(C)CN3)c2n1 ; C[C@@H](Nc1ccn2ncc(C3=NOCCN3)c2n1)c1cc(F)cc(F)c1F ; C[C@@H](Nc1ccn2ncc(C(=O)NOCCO)c2n1)c1cc(F)cc(F)c1F]

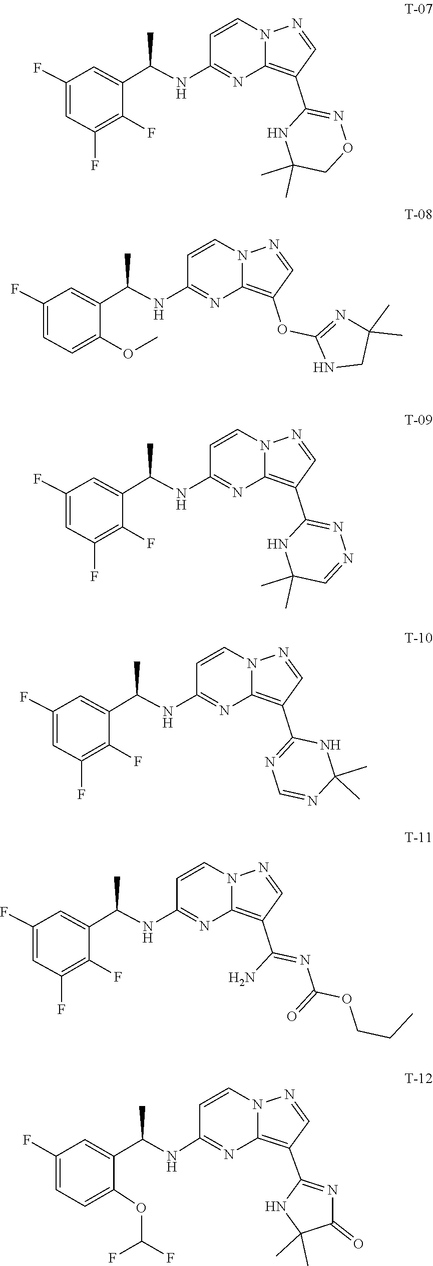

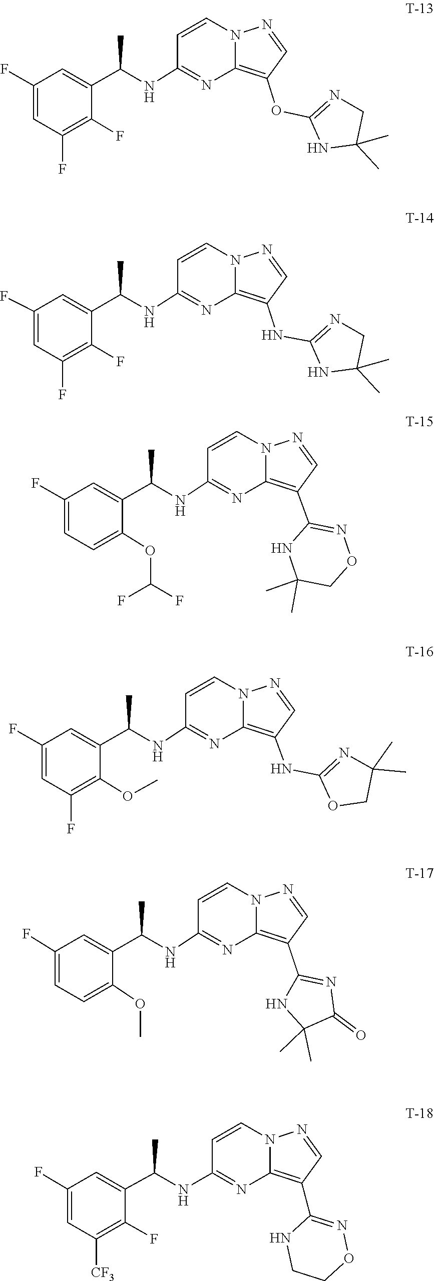 [SMILES: C[C@@H](Nc1ccn2ncc(OC3=NCC(C)(C)N3)c2n1)c1cc(F)cc(F)c1F ; C[C@@H](Nc1ccn2ncc(NC3=NCC(C)(C)N3)c2n1)c1cc(F)cc(F)c1F ; C[C@@H](Nc1ccn2ncc(C3=NOCC(C)(C)N3)c2n1)c1cc(F)ccc1OC(F)F ; COc1c(F)cc(F)cc1[C@@H](C)Nc1ccn2ncc(NC3=NC(C)(C)CO3)c2n1 ; COc1ccc(F)cc1[C@@H](C)Nc1ccn2ncc(C3=NC(=O)C(C)(C)N3)c2n1 ; C[C@@H](Nc1ccn2ncc(C3=NOCCN3)c2n1)c1cc(F)cc(C(F)(F)F)c1F]

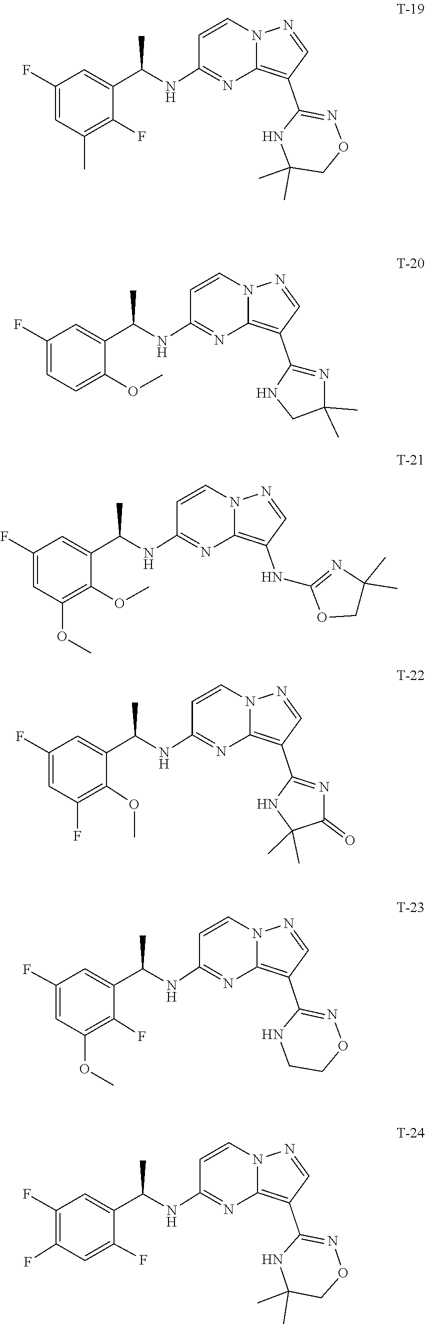 [SMILES: Cc1cc(F)cc([C@@H](C)Nc2ccn3ncc(C4=NOCC(C)(C)N4)c3n2)c1F ; COc1ccc(F)cc1[C@@H](C)Nc1ccn2ncc(C3=NC(C)(C)CN3)c2n1 ; COc1cc(F)cc([C@@H](C)Nc2ccn3ncc(NC4=NC(C)(C)CO4)c3n2)c1OC ; COc1c(F)cc(F)cc1[C@@H](C)Nc1ccn2ncc(C3=NC(=O)C(C)(C)N3)c2n1 ; COc1cc(F)cc([C@@H](C)Nc2ccn3ncc(C4=NOCCN4)c3n2)c1F ; C[C@@H](Nc1ccn2ncc(C3=NOCC(C)(C)N3)c2n1)c1cc(F)c(F)cc1F]

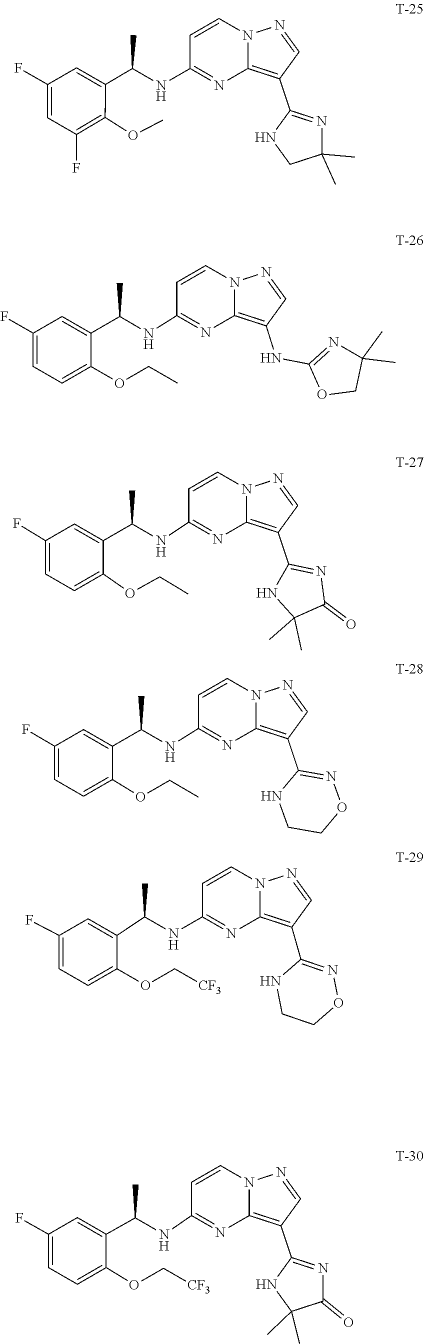

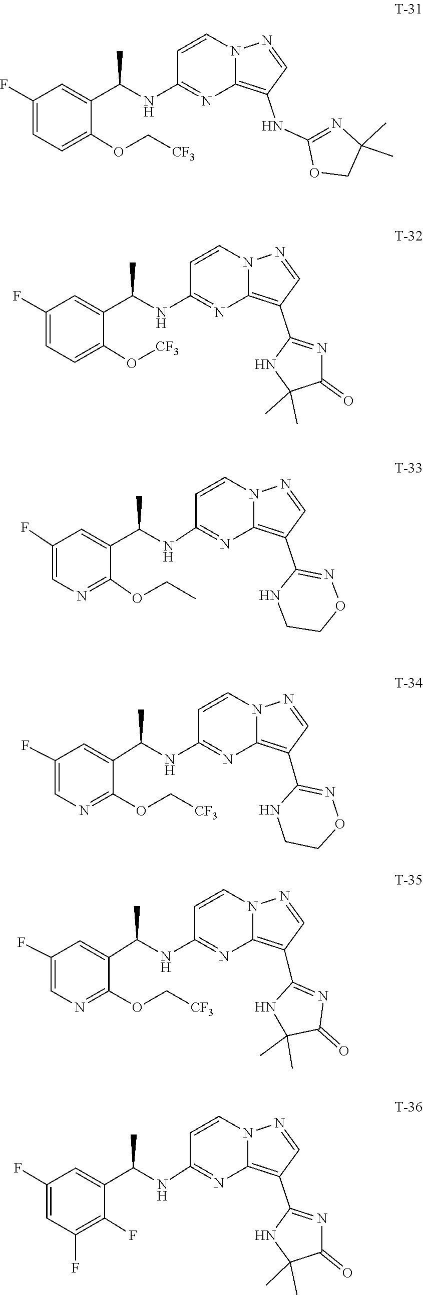 [SMILES: C[C@@H](Nc1ccn2ncc(NC3=NC(C)(C)CO3)c2n1)c1cc(F)ccc1OCC(F)(F)F ; C[C@@H](Nc1ccn2ncc(C3=NC(=O)C(C)(C)N3)c2n1)c1cc(F)ccc1OC(F)(F)F ; CCOc1ncc(F)cc1[C@@H](C)Nc1ccn2ncc(C3=NOCCN3)c2n1 ; C[C@@H](Nc1ccn2ncc(C3=NOCCN3)c2n1)c1cc(F)cnc1OCC(F)(F)F ; C[C@@H](Nc1ccn2ncc(C3=NC(=O)C(C)(C)N3)c2n1)c1cc(F)cnc1OCC(F)(F)F ; C[C@@H](Nc1ccn2ncc(C3=NC(=O)C(C)(C)N3)c2n1)c1cc(F)cc(F)c1F]

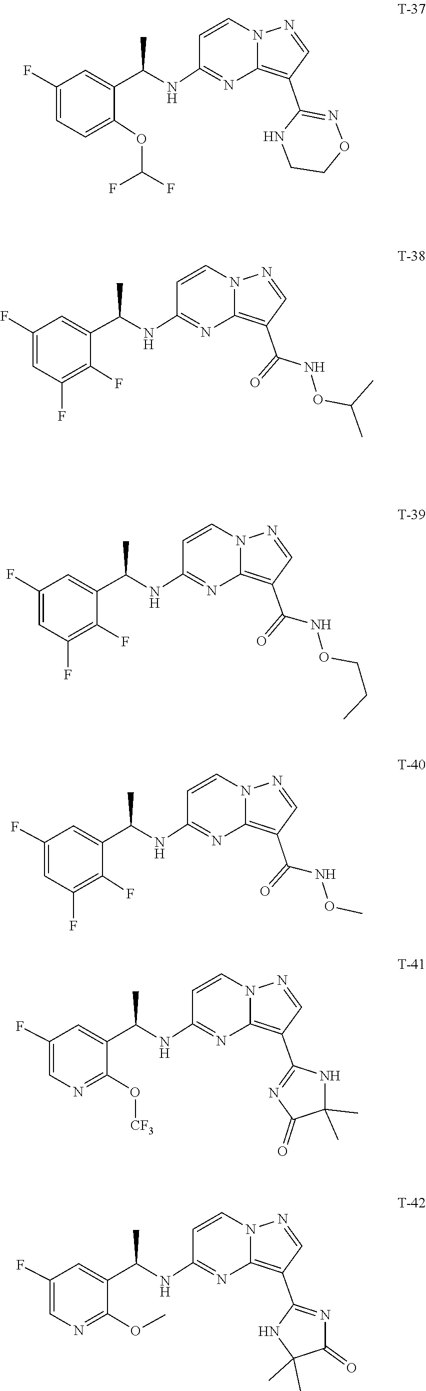 [SMILES: C[C@@H](Nc1ccn2ncc(C3=NOCCN3)c2n1)c1cc(F)ccc1OC(F)F ; CC(C)ONC(=O)c1cnn2ccc(N[C@H](C)c3cc(F)cc(F)c3F)nc12 ; CCCONC(=O)c1cnn2ccc(N[C@H](C)c3cc(F)cc(F)c3F)nc12 ; CONC(=O)c1cnn2ccc(N[C@H](C)c3cc(F)cc(F)c3F)nc12 ; C[C@@H](Nc1ccn2ncc(C3=NC(=O)C(C)(C)N3)c2n1)c1cc(F)cnc1OC(F)(F)F ; COc1ncc(F)cc1[C@@H](C)Nc1ccn2ncc(C3=NC(=O)C(C)(C)N3)c2n1]

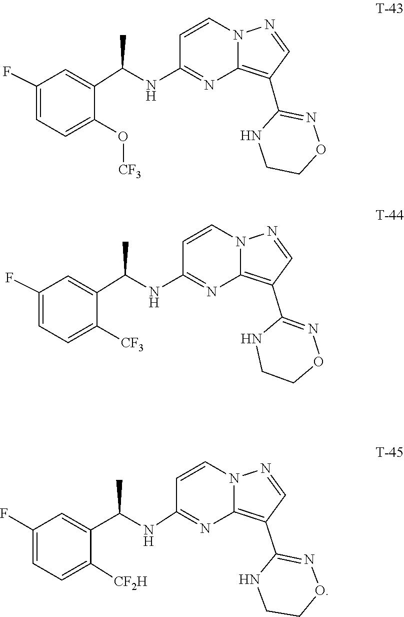 [SMILES: C[C@@H](Nc1ccn2ncc(C3=NOCCN3)c2n1)c1cc(F)ccc1OC(F)(F)F ; C[C@@H](Nc1ccn2ncc(C3=NOCCN3)c2n1)c1cc(F)ccc1C(F)(F)F ; C[C@@H](Nc1ccn2ncc(C3=NOCCN3)c2n1)c1cc(F)ccc1C(F)F]

In the second aspect of the present invention, it provides a pharmaceutical composition comprising i) a therapeutically effective amount of the compound of formula I, a stereoisomer, a tautomer, a crystalline form, a pharmaceutically acceptable salt, a hydrate, a solvate or a prodrug thereof of the first aspect of the present invention; and ii) one or more pharmaceutically acceptable carriers, diluents or excipients.

In another preferred embodiment, the pharmaceutical composition further comprises a drug selected from the group consisting of PD-1 inhibitor (e.g., nivolumab, pembrolizumab, pidilizumab, cemiplimab, JS-001, SHR-120, BGB-A317, IBI-308, GLS-010, GB-226, STW204, HX008, HLX10, BAT1306, AK105, LZM 009 or the biological analogue thereof, etc.), PD-L1 inhibitor (e.g, durvalumab, atezolizumab, avelumab, CS1001, KN035, HLX20, SHR-1316, BGB-A333, JS003, CS1003, KL-A167, F 520, GR1405, MSB2311 or the biological analogue thereof, etc.), CD20 antibody (e.g, rituximab, obinutuzumab, ofatumumab, veltuzumab, tositumomab, 131I-tositumomab, ibritumomab tiuxetan, 90Y-ibritumomab tiuxetan, 90In-ibritumomab tiuxetan, ibritumomab tiuxetan, etc.), CD47 antibody (e.g, Hu5F9-G4, CC-90002, TTI-621, TTI-622, OSE-172, SRF-231, ALX-148, NI-1701, SHR-1603, IBI188, IMM01), ALK inhibitor (e.g, Ceritinib, Alectinib, Brigatinib, Lorlatinib, Ocatinib), PI3K inhibitors (e.g, Idelalisib, Duvelisib, Dactolisib, Taselisib, Bimiralisib, Omipalisib, Buparlisib, etc.), BTK inhibitor (e.g, ibrutinib, Tirabrutinib, Acalabrutinib, Zanubrutinib, Vecabrutinib, etc.), EGFR inhibitor (e.g, Afatinib, Gefitinib, Erlotinib, Lapatinib, Dacomitinib, Icotinib, Canertinib, Sapitinib, Naquotinib, Pyrotinib, Rociletinib, Osimertinib, etc.), VEGFR inhibitor (e.g, Sorafenib, Pazopanib, Regorafenib, Sitravatinib, Ningetinib, Cabozantinib, Sunitinib, Donafenib, etc.), HDAC inhibitor (e.g, Givinostat, Tucidinostat, Vorinostat, Fimepinostat, Droxinostat, Entinostat, Dacinostat, Quisinostat, Tacedinaline, etc.), CDK inhibitor (e.g, Palbociclib, Ribociclib, Abemaciclib, etc.), MEK inhibitor (e.g, Selumetinib (AZD6244), Trametinib (GSK1120212), PD0325901, U0126, Pimasertib (AS-703026), PD184352 (CI-1040), etc.), mTOR inhibitor (e.g, Vistusertib, etc.), SHP2 inhibitor (e.g, RMC-4630, JAB-3068, TNO155, etc.), and a combination thereof.

In the third aspect of the present invention, it provides a method for preparing a pharmaceutical composition, comprising the step of mixing a pharmaceutically acceptable carrier with the compound or the stereoisomer or optical isomer, pharmaceutically acceptable salt, prodrug or solvate of the first aspect of the present invention, thereby forming the pharmaceutical composition.

In another preferred embodiment, the compound of the present invention can be prepared into powder, tablet, granule, capsule, solution, emulsion, suspension and the like.

In the fourth aspect of the present invention, it provides a use of the compound of formula I, or the stereoisomer, tautomer, crystalline form, pharmaceutically acceptable salt, hydrate, solvate or prodrug thereof of the first aspect of the present invention in the preparation of a medicament for preventing and/or treating the diseases with ROS1, NTRK or ALK-mediated pathological characteristics.

In another preferred embodiment, the diseases with ROS1, NTRK or ALK-mediated pathological characteristics include cancer, sarcoma and pain.

In another preferred embodiment, the cancer is selected from the group consisting of breast cancer, cervical cancer, colon cancer, lung cancer, stomach cancer, rectal cancer, pancreatic cancer, brain cancer, skin cancer, oral cancer, prostate cancer, bone cancer, kidney cancer, ovarian cancer, bladder cancer, liver cancer, fallopian tumor, peritoneal tumor, melanoma, glioma, glioblastoma, head and neck cancer, mastoid nephroma, leukemia, lymphoma, myeloma and thyroid tumor.

It should be understood that in the present invention, any of the technical features specifically described above and below (such as in the Example) can be combined with each other, thereby constituting new or preferred technical solutions. Limited by space, it will not be repeated here.

### DETAILED DESCRIPTION OF THE INVENTION

After extensive and in-depth research, the inventor of the present invention accidentally discovered a new compound having excellent inhibitory activity against ROS1, NTRK and ALK and their drug-resistant mutations, especially against drug-resistant mutations, and having better pharmacodynamics and pharmacokinetic properties and lower toxic and side effects. It has the potential to be developed into an effective drug for drug-resistant patients that is urgently needed in clinical practice.

### Term

Unless otherwise stated, the following terms used in this application (including the specification and claims) have the definitions given below.

“Alkyl”(alone or as part of other groups) refers to a monovalent linear or branched saturated hydrocarbon group composed only of carbon and hydrogen atoms. Wherein “C1-C6 alkyl” refers to alkyl containing 1 to 6 carbon atoms, preferably C1-C4 alkyl. Examples of alkyl include but are not limited to methyl, ethyl, propyl, isopropyl, isobutyl, sec-butyl, tert-butyl, amyl, n-hexyl, octyl and dodecyl etc. In the present invention, the alkyl is also intended to include a deuterated alkyl, examples of which include, but are not limited to CD₃, CD₂CD₃ and CD₂CD₂CD₃.

“Alkylene” (alone or as part of another group) refers to an alkyl as described above by removing a hydrogen atom, such as methylene (—CH₂—), ethylene (—CH₂CH₂—) and the like.

“Alkoxy” (alone or as part of another group) refers to the formula —OR or —R′—OR, wherein R is an alkyl as defined herein, and R′ is an alkylene. Examples of alkoxy include but are not limited to methoxy, ethoxy, isopropoxy, tert-butoxy, —CH₂O—CH₃, —CH₂CH₂—O—CH₃, —CH₂—O—CH₂CH₃, and the like.

“Halogen(halo)” (alone or as part of another group) refers to fluorine, chlorine, bromine or iodine.

“Haloalkyl” (alone or as part of other group) refers to a group which one or more hydrogens in an alkyl as defined above are replaced by the same or different halogens. Wherein “C1-C6 haloalkyl” is preferably C₁-C₄ haloalkyl, and examples of haloalkyl include, but are not limited to —CH₂Cl, —CH₂CF₃, —CH₂CCl₃, perfluoroalkyl (for example, —CF₃— and —CF₂CF₃), etc.

“Haloalkoxy” (alone or as part of another group) refers to a group of formula —OR, wherein R is a haloalkyl as defined herein. Examples of haloalkoxy include but are not limited to trifluoromethoxy, difluoromethoxy, and 2,2,2-trifluoroethoxy, etc.

“Cycloalkyl” (alone or as part of other group) refers to the single- or bicyclic monovalent saturated carbocyclic group only consists of carbon and hydrogen atoms, wherein “C3-C8 cycloalkyl” refers to a saturated carbocyclic group containing 3 to 8 carbon atoms, preferably C3-C6 cycloalkyl. Examples of cycloalkyl include, but are not limited to, cyclopropyl, cyclobutyl, cyclopentyl, cyclohexyl, and the like. The cycloalkyl may optionally be substituted with one or more substituents, wherein each substituent is independently a hydroxyl, alkyl, alkoxy, halogen, haloalkyl, amino, monoalkylamino or dialkylamino. Examples of cycloalkyl include but are not limited to cyclopropyl, cyclobutyl, cyclopentyl, cyclohexyl and cycloheptyl, etc.

“Cycloalkoxy” (alone or as part of another group) refers to a group of formula —OR, wherein R is cycloalkyl as defined herein. Examples of cycloalkyloxy include cyclopropyloxy, cyclobutyloxy, cyclopentyloxy and cyclohexyloxy, etc.

“Acyl” refers to a group of formula —C(O)R, wherein R is an alkyl or alkylamino as defined herein. “Acyl” is preferably —C(O)C₁-C₆ alkyl, —C(O)NH₂, —C(O)NHC₁-C₆ alkyl, —C(O)N(C₁-C₆ alkyl)₂, more preferably —C(O)C₁-C₃ alkyl, —C(O)NH₂, —C(O)NHC₁-C₃ alkyl, —C(O)N(C₁-C₃ alkyl)₂, examples of acyl include acetyl, n-propionyl, isopropionyl, n-butyryl, isobutyryl, tert-butyryl, —C(O)NH₂, —C(O)NHCH₃ and —C(O)N(CH)₃)₂, etc.

“Alkylamino” (alone or as part of other group) refers to a group of formula —NRaRb, wherein Ra and Rb are the same or different, and each independently H or alkyl as defined herein.

Ester group refers to a group of formula —C(O) OR, wherein R is an alkyl as defined herein. The ester group is preferably —C(O)OC₁-C₆ alkyl, more preferably —C(O)OC₁-C₄ alkyl, examples of ester group include —C(O)OMe, —C(O)OEt and —C(O)O—C(CH₃)₃, etc.

Sulfonyl refers to a group of formula —S(O)₂—R, wherein R is an alkyl as defined herein. The sulfonyl is preferably —S(O)₂—C₁-C₆ alkyl, examplarily comprising —S(O)₂-Me and —S(O)₂-Et, etc.

Sulfinyl refers to a group of formula —SO—R, wherein R is an alkyl as defined herein. Sulfinyl is preferably —SO—C₁-C₆ alkyl, examplarily comprising —SO-Me and —SO-Et, etc.

“Alkylthio” refers to a group of formula —SRa, wherein Ra is H or alkyl as defined herein.

“Cycloalkylamino” refers to a group of formula —NRaRb, wherein Ra is H, alkyl as defined herein, or cycloalkyl as defined herein, and Rb is cycloalkyl as defined herein.

“Heterocyclyl” (alone or as part of other group) refers to a completely saturated or partially unsaturated cyclic group (including but not limited to, for example, 3-7-membered monocyclic, 6-11-membered bicyclic, or 8-16-membered tricyclic system) in which at least one heteroatom is present in a ring having at least one carbon atom. Each heteroatom-containing heterocyclic ring has 1, 2, 3, or 4 heteroatoms selected from the group consisting of nitrogen, oxygen, or sulfur atoms, wherein the nitrogen or sulfur atoms may be oxidized or the nitrogen atoms may be quaternized.

Heterocycloalkyl refer to completely saturated heterocyclyl. Heterocyclyl can be attached to the residue of any heteroatom or carbon atom of the ring or ring molecule. Herein, “3-8 membered heterocyclyl” means a group having 3-8 ring members. Typical monocyclic heterocyclyls include, but are not limited to azetidinyl, pyrrolidyl, oxetanyl, pyrazolinyl, imidazolinyl, imidazolidinyl, oxazolidinyl, isoxazolidinyl, thiazolidinyl, isothiazolidinyl, tetrahydrofuryl, piperidyl, piperazinyl, 2-oxoppiperazinyl, 2-oxopiperidyl, 2-oxopyrrolidyl, hexahydroazepinyl, 4-piperidone, tetrahydropyranyl, morpholinyl, thiomorpholinyl, thiomorpholinylsulfoxide, thiomorpholinylsulfone, 1,3-dioxane and tetrahydro-1,1-dioxythienyl, etc. A polycyclic heterocyclyl includes spiro, fused, and bridged heterocyclyls. The spiro, fused, and bridged heterocyclyls involved are optionally connected with other groups by single bond, or are further fused with other cycloalkyl, heterocyclyl, aryl and heteroaryl by any two or more atoms of the ring.

“Aryl” (alone or as part of another group) refers to aromatic cyclic hydrocarbon groups with 1-5 rings, especially monocyclic and bicyclic groups. Any aromatic ring having two or more aromatic rings (bicyclic, etc.), the aromatic rings of aryl may be connected by single bond (such as biphenyl) or fused (such as naphthalene, anthracene, etc.), wherein, “C6-C12 aryl” refers to an aromatic cyclic hydrocarbon compound group containing 6, 7, 8, 9, 10, 11 or 12 ring carbon atoms. Examples of aryl (especially monocyclic and bicyclic groups) include but are not limited to phenyl, biphenyl or naphthyl. Aryl can be fused with heterocyclic groups through a single bond or any two adjacent ring C atoms, for example: benzotetrahydrofuranyl, benzotetrahydropyranyl, benzodioxanyl and

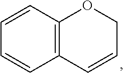

etc.

“Heteroaryl” (alone or as part of other groups) refers to heterocyclic aryl, wherein “5-12 membered heteroaryl” refers to a monocyclic, bicyclic or tricyclic aromatic ring group containing 5 to 12 ring atoms and containing at least 1 (such as 1, 2 or 3) ring heteroatoms selected from the group consisting of N, O or S, and the remaining ring atoms are C. It should be clear that the connection point of heteroaryl should be located on the heteroaromatic ring. Heteroaryl is preferred to have 5-8 ring atoms (5-8 membered), more preferably have 5-6 ring atoms (5-6 membered). Examples of heteroaryl include but are not limited to imidazolyl, aoxazolyl, isoxazolyl, thiazolyl, isothiazolyl, oxadiazolyl, thiadiazolyl, pyrazinyl, thienyl, furanyl, pyranyl, pyridyl, pyrrolyl, pyrazolyl, pyrimidinyl, quinolinyl, isoquinolinyl, benzofuranyl, benzothienyl, benzothiopyranyl, benzimidazolyl, benzoxazolyl, benzoxadiazolyl, benzothiazolyl, benzothiadiazolyl, benzopyranyl, indolyl, isoindolyl, triazolyl, triazinyl, quinoxalinyl, purinyl, quinazolinyl, quinazinyl, naphthyridinyl, pterridinyl, carbazolyl, azepinyl, diazepinyl and acridinyl, etc.

“Polysubstituted” means substituted by two or more substituents.

In the present invention, unless other stated, the alkyl, cycloalkyl, heterocyclyl, aryl, heteroaryl and other groups include substituted alkyl, cycloalkyl, heterocyclyl, aryl, heteroaryl, etc., the substituents such as (but not limited to) halogen, hydroxyl, cyano, acyl, sulfonyl, ester, sulfinyl, alkyl, cycloalkyl, heterocyclyl, aryl, heteroaryl, acyl, ester, etc.

“Deuterated compound” refers to the compound obtained by replacing one hydrogen atom (H) or multiple hydrogen atoms (H) with deuterium atoms (D) in a compound.

Active Ingredient

As used herein, the terms “compound of the present invention” or “active ingredient of the present invention” are used interchangeably and refer to a compound of formula I, a stereoisomer, a tautomer, a crystalline form, a pharmaceutically acceptable salt, a hydrate, a solvate, or a prodrug thereof.

A compound of formula I, or a stereoisomer, a tautomer, a crystalline form, a pharmaceutically acceptable salt, a hydrate, a solvate or a prodrug thereof has the following structure:

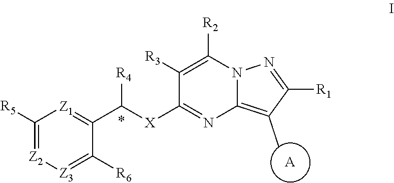

wherein,

* is R or S configuration;

A, Z₁, Z₂, Z₃, R₁, R₂, R₃, R₄, R₅, R₆ and X are as defined above.

Preferably, the compound of formula I has the structure shown in formula II:

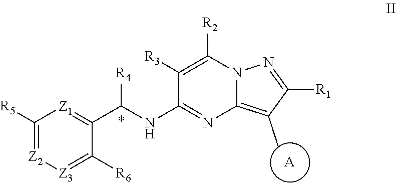

wherein,

* is R or S configuration;

Z₁, Z₂, Z₃, R₁, R₂, R₃, R₄, R₅, R₆ and A are as defined above.

Preferably, the compound of formula I has the structure shown in formula III or IV:

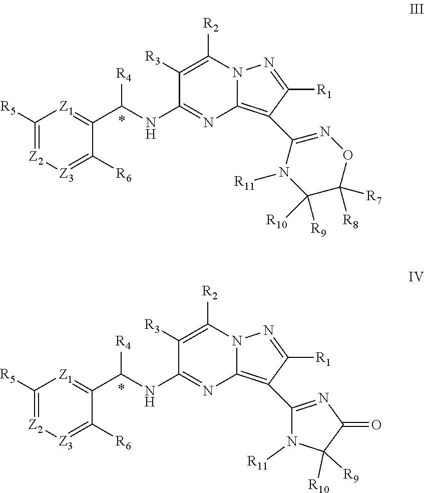

wherein,

* is R or S configuration;

Z₁, Z₂, Z₃, R₁, R₂, R₃, R₄, R₅, R₆, R₇, R₈, R₉, R₁₀ and Ru are as defined above.

Preferably, the compound of formula I has the structure shown in formula V or VI:

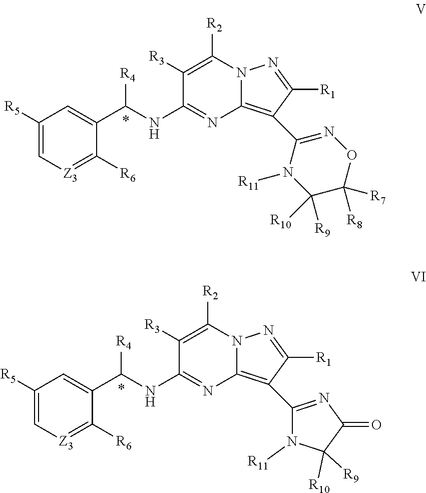

wherein,

* is R or S configuration;

R₄ is selected from the group consisting of H, C1-C6 alkyl, C3-C6 cycloalkyl and C1-C6 haloalkyl;

Z₃, R₁, R₂, R₃, R₅, R₆, R₇, R₈, R₉, R₁₀ and Ru are as defined above.

Preferably it has the structure shown in formula VII or VIII:

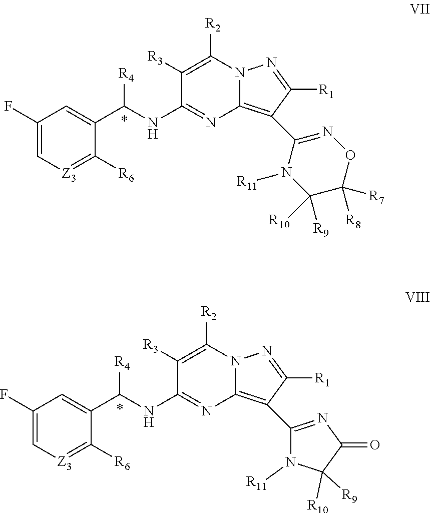

wherein,

* is R or S configuration;

Z₃, R₁, R₂, R₃, R₄, R₆, R₇, R₈, R₉, R₁₀ and Ru are as defined above.

Preferably, in formula I-VIII, R₆ is selected from the group consisting of halogen, C1-C3 haloalkoxy and C1-C6 haloalkylamino.

Preferably, in the formula I-VIII, R₁, R₂ and R₃ are each independently selected from the group consisting of hydrogen, halogen and amino;

R₄ is selected from the group consisting of hydrogen, C1-C6 alkyl, C3-C6 cycloalkyl, and C1-C6 haloalkyl;

R₅ is selected from the group consisting of hydrogen and halogen;

R₆ is selected from the group consisting of hydrogen, halogen, C1-C6 alkyl, C3-C6 cycloalkyl, C1-C6 alkoxy, C1-C6 alkylamino, C1-C6 haloalkyl, C1-C6 haloalkoxy, C1-C6 haloalkylamino and C3-C6 halocycloalkyl.

Preferably, in formula III-VIII, R₇, R₈, R₉ and R₁₀ are each independently selected from the group consisting of hydrogen, C1-C3 alkyl and C1-C3 haloalkyl.

Preferably, in formula I-IV, Z₁ is CR₁₃, preferably CH.

Preferably, in formula I-IV, Z₂ is CR₁₃, preferably CH.

Preferably, in the formula III-VIII, R₁, R₂ and R₃ are each independently selected from the group consisting of hydrogen, halogen and amino;

R₄ is selected from the group consisting of hydrogen, C1-C6 alkyl, C3-C6 cycloalkyl, and C1-C6 haloalkyl;

R₅ is selected from the group consisting of hydrogen and halogen;

R₆ is selected from the group consisting of hydrogen, halogen, C1-C6 alkyl, C3-C6 cycloalkyl, C1-C6 alkoxy, C1-C6 alkylamino, C1-C6 haloalkyl, C1-C6 haloalkoxy, C1-C6 haloalkylamino and C3-C6 halocycloalkyl;

R₇, R₈, R₉, R₁₀ and R₁ are each independently selected from the group consisting of hydrogen and substituted or unsubstituted C1-C6 alkyl; wherein, the “substituted” refers to being substituted by one or more groups selected from the group consisting of deuterium, halogen, amino, cyano, hydroxyl, acyl, ester group, C1-C6 alkyl, C1-C6 haloalkyl, C3-C6 cycloalkyl, C3-C6 halocycloalkyl, C1-C6 alkoxy, C1-C6 haloalkoxy, C6-C10 aryl and 5-10 membered heteroaryl.

The salt that the compound in the present invention may be formed are also within the scope of the present invention. Unless otherwise stated, the compound in the present invention is understood to include its salt. The term “salt” as used herein refers to a salt formed in the form of acid or base from inorganic or organic acid and base. Further, when the compound in the present invention contains a base fragment which includes, but is not limited to pyridine or imidazole, when contains an acid fragment which includes, but is not limited to carboxylic acid. The zwitter-ion that may form “inner salt” is included within the scope of the term “salt”. Pharmaceutically acceptable (i.e., non-toxic, physiologically acceptable) salt is preferred, although other salts are also useful and may be used, for example, in the separation or purification steps of the preparation process. The compound of the present invention may form a salt, for example, compound I is reacted with a certain amount (such as an equivalent amount) of an acid or base, and precipitated in a medium, or freeze-dried in aqueous solution.

The base fragment contained in the compounds of the present invention includes but is not limited to amines or pyridine or imidazole ring, which may form salt with organic or inorganic acid. Typical acids that can form salts include hydrochloride, hydrobromide, hydroiodate, sulfate, bisulfate, nitrate, phosphate and acid phosphate; the organic acid salt is selected from formate, acetate, trifluoroacetate, propionate, pyruvate, hydroxyacetate, oxalate, malonate, fumarate, maleate, lactate, malate, citrate, tartrate, methanesulfonate, ethanesulfonate, hydroxyethanesulfonate, benzenesulfonate, salicylate, picrate, glutamate, ascorbate, camphorate, camphor sulfonate, etc.

Acidic fragments that may be contained in some compounds of the invention includes, but not limited to carboxylic acid, which may form salts with various organic or inorganic bases. Salt formed by typical base includes ammonium salt, alkali metal salt (such as sodium, lithium and potassium salts), alkaline earth metal salt (such as calcium and magnesium salts), and salt formed by organic bases (such as organic amines), such as benzathine, dicyclohexylamine, hydrabamine (salt formed with N,N-bis(dehydroabietyl)ethylenediamine), N-methyl-D-glucanamine, N-methyl-D-glucoamide, tert-butyllamine, and the salt formed with amino acids such as arginine, lysine, etc. Basic nitrogen-containing groups can form quaternary ammonium salts with halides, such as small molecular alkyl halides (such as chlorides, bromides and iodides of methyl, ethyl, propyl and butyl), dialkyl sulfate (such as dimethyl, diethyl, dibutyl, and dipentyl sulfates), long chain halides (such as chlorides, bromides and iodides of decyl, dodecyl, tetradecyl, and tetradecyl), aralkyl halides (such as bromides of benzyl and phenyl), etc.

The prodrug and solvate of the compound in the present invention are also included within the scope of the present invention. The term “prodrug” herein refers to a compound resulting from the chemical transformation of a metabolic or chemical process to produce a compound, salt, or solvate in the present invention for the treatment of an associated disease. The compound of the invention includes solvate such as hydrate.

Compound, salt or solvate in the present invention, may be present in tautomeric forms such as amide and imino ether. All of these tautomers are part of the present invention.

Stereisomers of all compounds (e.g., those asymmetric carbon atoms that may be present due to various substitutions), including their enantiomeric forms and non-enantiomed forms, all belong to the protection scope of the present invention. The independent stereoisomer in the present invention may not coexist with other isomers (e.g., as a pure or substantially pure optical isomer with special activity), or may be a mixture (e.g., racemate), or a mixture formed with all other stereoisomers or a part thereof. The chiral center of the present invention has two configurations of S or R, which is defined by International Union of Pure and Applied Chemistry (IUPAC) in 1974. The racemization form can be solved by physical methods, such as fractional crystallization, or separation crystallization by derivation into diastereomers, or separation by chiral column chromatography. Individual optical isomer can be obtained from racemate by appropriate methods, including but not limited to conventional methods, such as recrystallization after salting with optically active acids.

Weight content of compound in the present invention obtained by preparation, separation and purification in turn, and is equal to or greater than 90%, such as equal to or greater than 95%, equal to or greater than 99% (“very pure” compound), and listed in the description of the text. In addition, the “very pure” compound of the present invention is also part of the present invention.

All configuration isomers of the compound of the present invention are within the scope, whether in mixture, pure or very pure form. The definition of the compound of the present invention comprises cis (Z) and trans (E) olefin isomers, and cis and trans isomers of carbocyclic and heterocyclic.

In the entire specification, the groups and substituents can be selected to provide stable fragments and compounds.

Specific functional groups and chemical term definitions are described as follows in detail. For the purposes of the present invention, the chemical elements are consistent with Periodic Table of the Elements, CAS version, Handbook of Chemistry and Physics, 75^{th} Ed. The definition of a particular functional group is also described therein. In addition, the basic principles of Organic Chemistry as well as specific functional groups and reactivity described in “Organic Chemistry”, Thomas Sorrell, University Science Books, Sausalito: 1999, the entire content of which is incorporated herein by reference.

Some compounds of the present invention may exist in specific geometric or stereoisomer forms. The present invention covers all compounds, including their cis and trans isomers, R and S enantiomers, diastereomers, (D) type isomers, (L) type isomers, racemic mixtures and other mixtures. In addition, asymmetric carbon atom can represent substituent, such as alkyl. All isomers and mixtures thereof are included in the present invention.

According to the invention, mixtures of isomers may contain a variety ratio of isomers. For example, mixtures with only two isomers may have the following combinations: 50:50, 60:40, 70:30, 80:20, 90:10, 95:5, 96:4, 97:3, 98:2, 99:1, or 100:0, all ratios of the isomers are within the scope of the present invention. Similar ratios readily understood by those of ordinary skill in the art and ratios for mixtures of more complex isomers are also within the scope of the present invention.

The invention also includes isotope labeled compounds, which are equivalent to the original compounds disclosed herein. However, in practice, it usually occurs when one or more atoms are replaced by atoms with a different atomic weight or mass number. Examples of compound isotopes that may be listed in the present invention include hydrogen, carbon, nitrogen, oxygen, phosphorus, sulfur, fluorine and chlorine isotopes, such as ²H, ³H, ¹³C, ¹¹C, ¹⁴C, ¹⁵N, ¹⁸O, ¹⁷O, ³¹P, ³²P, ³⁵S, ¹⁸F and ³⁶Cl. The compound, or enantiomer, diastereomer, isomer, or pharmaceutically acceptable salt or solvate, wherein the compound containing isotopes or other isotope atoms of above compound are all within the scope of the invention. Some isotope-labeled compounds in the present invention, such as the radioactive isotopes of ³H and ¹⁴C, are also included and are useful in experiments on the tissue distribution of drugs and substrates. Tritium (³H) and Carbon-14 (¹⁴C), which are relatively easy to prepare and detect and are the preferred choice. In addition, heavier isotope substitutions such as deuterium, i.e. ²H, have advantages in certain therapies due to their good metabolic stability, such as increased half-life or reduced dosage in vivo, and thus may be preferred in certain situations. Isotope-labeled compounds can be prepared by conventional methods through replacing readily available isotope-labeled reagents with non-isotopic reagents that can be prepared using the disclosed scheme shown in the Example.

If the synthesis of a specific enantiomer of the compound of the invention is to be designed, it can be prepared by asymmetric synthesis, or derivatized with chiral adjuvant, separating the resulting diastereomeric mixture and removing the chiral adjuvant to obtain a pure enantiomer. In addition, if a molecule contains a basic functional group, such as an amino acid, or an acidic functional group, such as a carboxyl group, a diastereomer salt can be formed with a suitable optically active acids or bases, which can be separated by conventional means, such as crystallization or chromatography, to obtain a pure enantiomer.

As described herein, the compound in the present invention may be substituted with any number of substituents or functional groups to extend its scope. In general, whether the term “substituted” appears before or after the term “optional”, the general formula that includes substituents in the compound of the present invention means the substitution of a specified structural substituent for a hydrogen radical. When multiple locations in a particular structure are replaced by multiple specific substituents, each location of the substituents can be the same or different. The term “substituted” as used herein includes all substitution that allows organic compounds to be substituted. Broadly speaking, the allowable substituents include non-cyclic, cyclic, branched, non-branched, carbocyclic and heterocyclic, aromatic ring and non-aromatic organic compounds. In the present invention, for example, heteroatom nitrogen, its valence state may be supplemented by a hydrogen substituent or by any permitted organic compound described above. Furthermore, the invention is unintentionally limited to the substituted organic compounds. The present invention considers that a combination of substituents and variable groups is good for the treatment of diseases in the form of stable compounds. The term “stable” herein refers to a stable compound which is sufficient for maintaining the integrity of the compound structure within a sufficiently long time, preferably in a sufficiently long time, which is hereby used for the above purposes.

The metabolites of the compounds of the present application and their pharmaceutically acceptable salts, and prodrugs that can be converted into the compounds of the present application and their pharmaceutically acceptable salts thereof in vivo, also included in the claims.

Preparation Method

The compound of the invention may be conveniently prepared by optionally combining the various synthetic methods described in this specification or known in the art, such a combination may be easily performed by a skilled person in the art to which the invention belongs.

Generally, in the preparation process, each reaction is usually carried out in an inert solvent at −60° C. to 100° C., preferably −60° C. to 80° C. The reaction time is usually 0.1-60 hours, preferably 0.5-48 hours.

The preferred synthetic route is as follows:

Scheme 1:

(1) in an inert solvent (such as ethanol and methanol), compound 1 and compound 2 undergo a nucleophilic substitution reaction in the presence of a base (such as sodium carbonate, potassium carbonate, sodium hydroxide, triethylamine, pyridine, etc.) to obtain compound 3;

(2) in an inert solvent (such as ethanol and methanol), compound 3 is reduced to obtain compound 4;

(3) in an inert solvent (such as 1,2-dichloroethane and/or tetrahydrofuran), compound 4 is reacted with 1,1-thiocarbonyldiimidazole and amino alcohol raw materials to obtain compound 5;

(4) in an inert solvent (such as 1,2-dichloroethane and/or tetrahydrofuran) and in the role of a base, compound 5 is reacted to obtain the final compound of formula I;

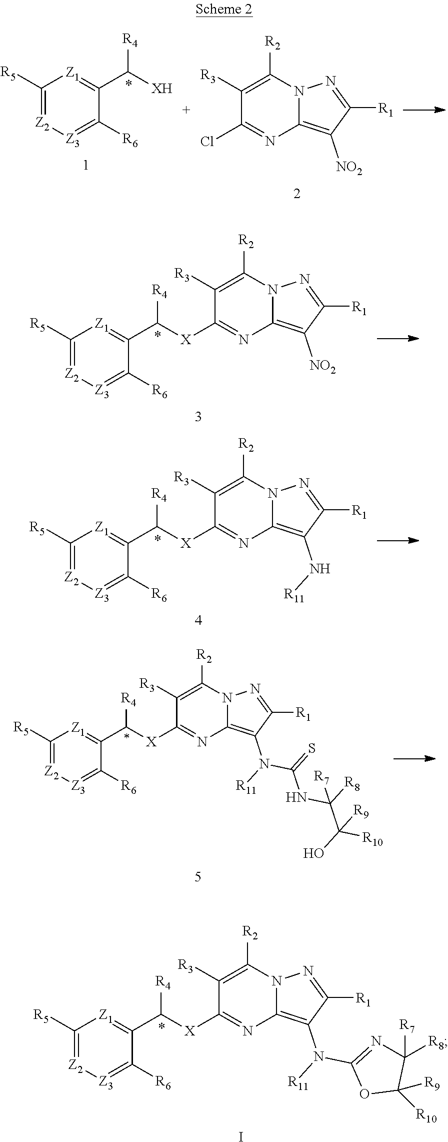

(1) in an inert solvent (such as ethanol and methanol), compound 1 and compound 2 undergo a nucleophilic substitution reaction in the presence of a base (such as sodium carbonate, potassium carbonate, sodium hydroxide, triethylamine, pyridine, etc.) to obtain compound 3;

(2) in an inert solvent (such as ethanol and methanol), compound 3 is reduced to obtain compound 4;

(3) in an inert solvent (such as 1,2-dichloroethane and/or tetrahydrofuran) and in the presence of a base, compound 4 is reacted to obtain the final compound of formula I.

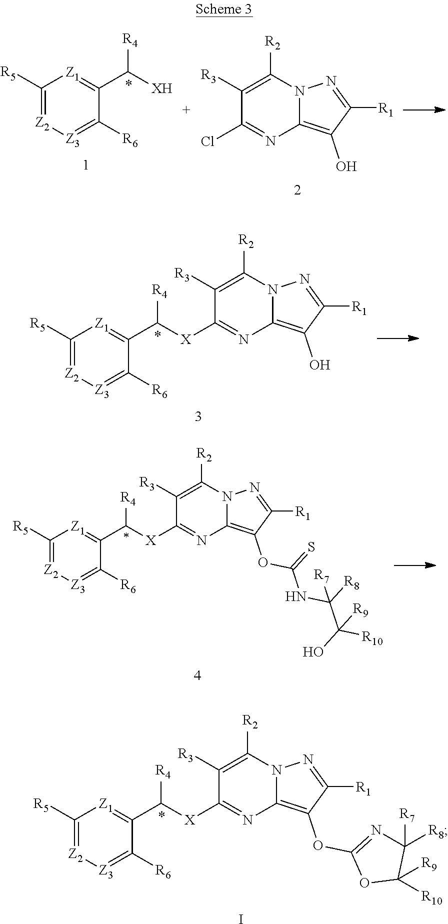

(1) in an inert solvent (such as ethanol or methanol), compound 1 and compound 2 undergo a nucleophilic substitution reaction in the presence of a base (such as sodium tert-butoxide, potassium tert-butoxide, sodium hydride, potassium hydride, potassium carbonate, cesium carbonate, potassium phosphate, potassium hydroxide, sodium hydroxide, etc.) to obtain compound 3;

(2) in an inert solvent (such as ethanol) and under acidic conditions, it is reacted to obtain compound 4;

(3) in an inert solvent (such as toluene or xylene) and under acid catalysis, compound 4 reacts with amino acid raw materials to obtain the final compound of formula I

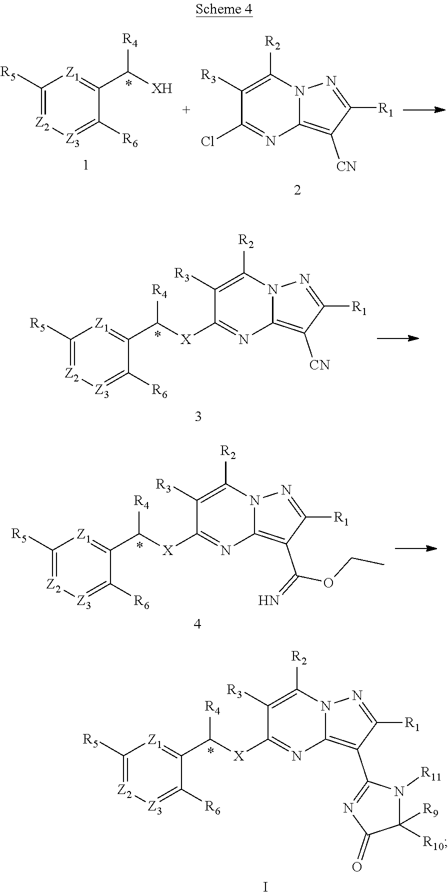

(1) in an inert solvent (such as ethanol or methanol), compound 1 and compound 2 undergo a nucleophilic substitution reaction in the presence of a base (such as sodium tert-butoxide, potassium tert-butoxide, sodium hydride, potassium hydride, potassium carbonate, cesium carbonate, potassium phosphate, potassium hydroxide, sodium hydroxide, etc.) to obtain compound 3;

(2) in an inert solvent (such as ethanol), under alkaline conditions, compound 3 reacts with hydroxylamine hydrochloride to obtain compound 4;

(3) in an inert solvent (such as toluene or xylene), compound 4 reacts with the corresponding raw material to obtain the final compound of formula I

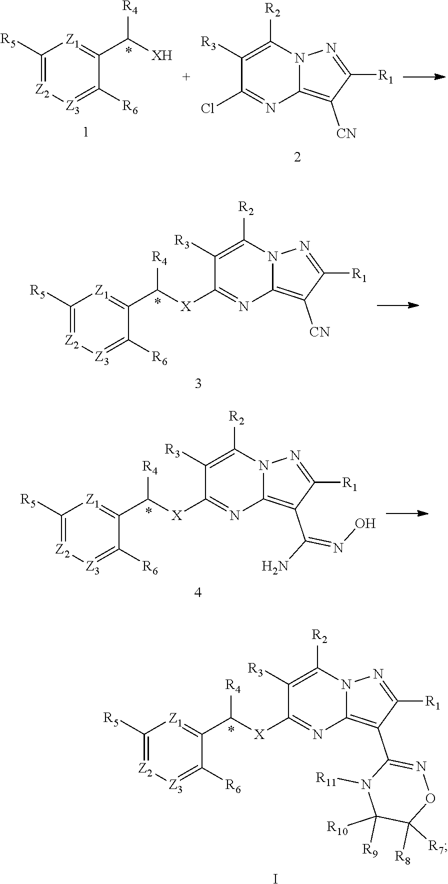

wherein,

Z₁, Z₂, Z₃, R₁, R₂, R₃, R₄, R₅, R₆, R₇, R₈, R₉, R₁₀, Ru and X are as defined above.

The starting materials of the present invention are known and commercially available, or can be synthesized by or according to the literature reported in the art.

Pharmaceutical Composition and Method of Administration

The pharmaceutical compositions of the present invention are used to prevent and/or treat the following diseases: inflammation, cancer, cardiovascular disease, infection, immunological disease, metabolic disease.

The compounds of the present invention can be used in combination with other drugs known to treat or improve similar conditions. When administered in combination, the original administration for the drug can remain unchanged, while compound of the present invention may be administered simultaneously or subsequently. Pharmaceutical composition containing one or more known drugs and the compound of the present invention may be preferred when administered in combination with one or more other drugs. The drug combination also includes administering the compound of the present invention and other one or more known drugs at overlapping time. When the compound of the present invention is combined with other one or more drugs, the dose of the compound of the present invention or known drug may be lower than that of their individual use.

The dosage forms of the pharmaceutical composition of the present invention include (but are not limited to): injection, tablet, capsule, aerosol, suppository, pellicle, pill, liniment for external use, controlled release or sustained-release or nano formulation.

The pharmaceutical composition of the present invention comprises a compound of the present invention or a pharmaceutically acceptable salt and a pharmaceutically acceptable excipient or carrier with safe and effective amount, wherein “safe and effective amount” refers to the amount of compound is sufficient to significantly improve the condition, not to produce severe side effects. Typically, the pharmaceutical composition contains 1-2000 mg of the compound/dosage of the present invention, and preferrably contains 10-1000 mg of the compound/dosage of the present invention. Preferably, “one dosage” is a capsule or a pill.

“Pharmaceutically acceptable carrier” refers to one or more compatible solid or liquid filler or gel substances, which are suitable for human use, and must be sufficiently pure and of sufficiently low toxicity. “Compatible” herein refers to ability of each component of a composition can be mixed with the compound of the present invention and can be mixed with each other without appreciably reducing the efficacy of the compound. Examples of pharmaceutically acceptable carrier include cellulose and derivatives thereof (such as sodium carboxymethylcellulose, sodium ethylcellulose, cellulose acetate, etc.), gelatin, talc, solid lubricant (such as stearic acid, magnesium stearate), calcium sulfate, vegetable oil (such as soybean oil, sesame oil, peanut oil, olive oil, etc.), polyol (such as propylene glycol, glycerol, mannitol, sorbitol, etc.), emulsifier (such as Tween®), wetting agent (such as lauryl sodium sulfate), colorant, flavoring, stabilizer, antioxidant, preservative, pyrogen-free water, etc.

There is no special limitation of administration mode for the compound or pharmaceutical compositions of the present invention, and the representative administration mode includes (but is not limited to): oral, intratumoral, rectal, parenteral (intravenous, intramuscular or subcutaneous), and topical administration.

Solid dosage forms for oral administration include capsules, tablets, pills, powders and granules. In these solid dosage forms, the active compounds are mixed with at least one conventional inert excipient (or carrier), such as sodium citrate or dicalcium phosphate, or mixed with any of the following components: (a) fillers or compatibilizer, such as starch, lactose, sucrose, glucose, mannitol and silicic acid; (b) binders, such as hydroxymethyl cellulose, alginate, gelatin, polyvinylpyrrolidone, sucrose and arabic gum; (c) humectant, such as, glycerol; (d) disintegrating agent, such as agar, calcium carbonate, potato starch or tapioca starch, alginic acid, certain composite silicates, and sodium carbonate; (e) dissolution-retarding agents, such as paraffin; (f) absorption accelerators, such as quaternary ammonium compounds; (g) wetting agents, such as cetyl alcohol and glyceryl monostearate; (h) adsorbents, for example, kaolin; and (i) lubricants such as talc, calcium stearate, magnesium stearate, solid polyethylene glycol, lauryl sodium sulfate, or the mixtures thereof. In capsules, tablets and pills, the dosage forms may also contain buffering agents.

The solid dosage forms such as tablets, sugar pills, capsules, pills and granules can be prepared by using coating and shell materials, such as enteric coatings and any other materials known in the art. They can contain an opaque agent. The release of the active compounds or compounds in the compositions can be released in a delayed mode in a given portion of the digestive tract. Examples of the embedding components include polymers and waxes. If necessary, the active compounds and one or more above excipients can form microcapsules.

Liquid dosage forms for oral administration include pharmaceutically acceptable emulsions, solutions, suspensions, syrups or tinctures. In addition to the active compounds, the liquid dosage forms may contain any conventional inert diluents known in the art such as water or other solvents, solubilizers and emulsifiers, such as ethanol, isopropanol, ethyl carbonate, ethyl acetate, propylene glycol, 1,3-butanediol, dimethyl formamide, as well as oil, in particular, cottonseed oil, peanut oil, corn germ oil, olive oil, castor oil and sesame oil, or the combination thereof.

Besides these inert diluents, the composition may also contain additives such as wetting agents, emulsifiers, and suspending agent, sweetener, flavoring agents and perfume.

In addition to the active compounds, the suspension may contain suspending agent, for example, ethoxylated isooctadecanol, polyoxyethylene sorbitol and sorbitan esters, microcrystalline cellulose, methanol aluminum and agar, or the combination thereof.

The compositions for parenteral injection may comprise physiologically acceptable sterile aqueous or anhydrous solutions, dispersions, suspensions or emulsions, and sterile powders which can be re-dissolved into sterile injectable solutions or dispersions. Suitable aqueous and non-aqueous carriers, diluents, solvents or excipients include water, ethanol, polyols and any suitable mixtures thereof.

The dosage forms for topical administration of compounds of the invention include ointments, powders, patches, aerosol, and inhalants. The active ingredients are mixed with physiologically acceptable carriers and any preservatives, buffers, or propellant if necessary, under sterile conditions.

Compounds of the present invention can be administrated alone, or in combination with other treatment means or therapeutic drugs.

When the pharmaceutical compositions are used, a safe and effective amount of compound of the present invention is administrated to a mammal (such as human) in need thereof, wherein the dose of administration is a pharmaceutically effective dose. For a person weighed 60 kg, the daily dose is usually 1-2000 mg, preferably 10-1000 mg. Of course, the particular dose should also depend on various factors, such as the route of administration, patient healthy status, which are all within the skills of an experienced physician.

The present invention also provides a preparation method of pharmaceutical composition comprising the step of mixing a pharmaceutically acceptable carrier with the compound or the pharmaceutically acceptable salt, stereoisomer, solvate or prodrug thereof of the present invention, thus forming the pharmaceutical composition.

The invention also provides a treatment method comprising the steps of administering the compound, or a pharmaceutically acceptable salt, a stereoisomer, a solvate or a prodrug thereof, or administering the pharmaceutical composition of the invention to a subject in need thereof to selectively inhibit fusion mutations and drug resistance mutations of ROS1, NTRK and ALK, etc.

The invention has the following main advantages:

(1) The compound of the invention has good inhibition ability to ROS1, NTRK and ALK kinase, especially excellent activity to drug-resistant mutation of these targets.

(2) The compound of the invention has better pharmacodynamics, pharmacokinetic properties and lower toxic and side effects.

(3) The compound of the invention has great potential to be developed into an effective drug for drug-resistant patients urgently needed clinically at present.

The technical solution of the present invention will be further described below, but the scope of protection of the present invention is not limited thereto.

Some specific examples are listed below for explanation.

### Example 1

Synthetic Route:

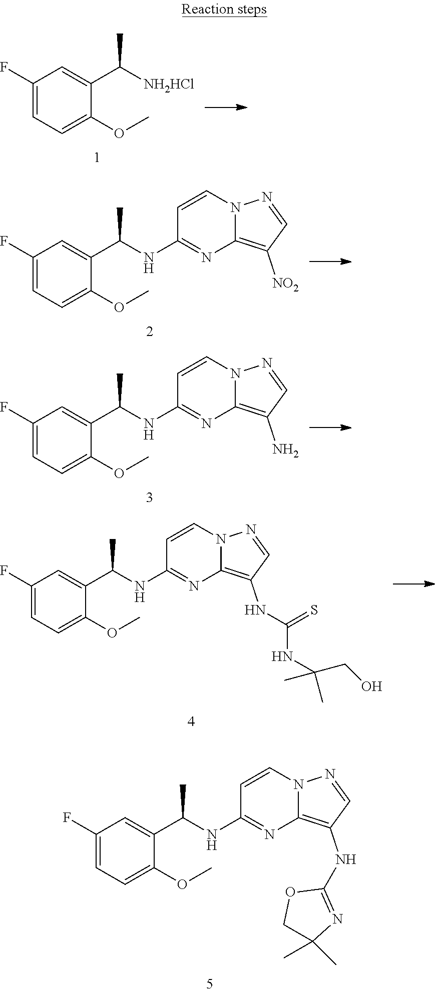

### (1) Synthesis of Compound 2

Compound 1 (1 g, 1 eq) was dissolved in a mixed solvent of EtOH:THF=24 ml:6 ml, and 5-chloro-3-nitropyrazolo [1,5-a]pyrimidine(1.01 g, 1.05 eq) and DIEA(1.9 g, 3 eq) were added to react at 55° C. for 4 h under N2 protection. After the reaction was completed, the mixture was mixed with silica gel and purified by column chromatography to obtain 1.5 g of compound 2.

### (2) Synthesis of Compound 3

Compound 2 (1.5 g, 1 eq) was added to 45 ml EtOH, Fe powder (0.76 g, 3 eq) and NH4Cl(0.7 g, 3 eq) were added to react at 85° C. for 3 h under the protection of N2. After the reaction was completed, the mixture was filtered, and EtOH was spin-dried. Water and EA were added for extraction, and the mixture was spin-dried to obtain 1.4 g of compound 3, which was directly used in the next step.

### (3) Synthesis of Compound 4

Under the protection of N₂, 1,1′-thiocarbonyldiimidazole (0.065 g, 1.1 eq) was dissolved in 1.5 ml THE at −10° C., then a solution of compound 3 (0.1 g, 1 eq) in 1.5 ml THE was added, and the reaction was carried out for 5 min at this temperature, then 0.5 ml solution of 2-amino-2-methylpropan-1-ol(0.04 g, 1.3 eq) was dropped into the reaction system to react at room temperature overnight. After the reaction was completed, the mixture was mixed with silica gel and purified by column chromatography to obtain 0.15 g of compound 4

### (4) Example 1: Synthesis of Compound T-01 (Compound 5)

Compound 4 (0.15 g, 1 eq) was added to 5 ml THF, NaOH(0.09 g, 6 eq) was added, and then TosCl(0.06 g, 0.9 eq) was added to react at room temperature for 3 h under the protection of N2. After the reaction was completed, the mixture was added with water and EA for extraction, and spin-dried to obtain 0.18 g of compound 5.

Synthesis of Compound Example 1: compound 7 (0.411 g) was weighted, dimethoxy acetonide (0.457 g, 4 eq), 1,2-dichloroethane (15 ml) and glacial acetic acid (7.5 ml) were added, stirred at 80° C. for 4 hours. The reaction was monitored by TLC until it was completed. The solvent was directly evaporated, then water and dichloromethane were added for extraction, and the mixture was dried, spin-dried and purified by column chromatography to obtain 170 mg of final product. 1H NMR (400 MHz, CDCl3) δ 8.25-8.12 (m, 2H), 7.05 (dd, J=9.1, 8.2 Hz, 1H), 6.80 (dd, J=9.1, 4.0 Hz, 1H), 6.08 (t, J=30.1 Hz, 4H), 3.91 (s, 2H), 1.58 (t, J=5.8 Hz, 8H), [M+H]⁺=399.2.

### Example 2

Synthetic Route:

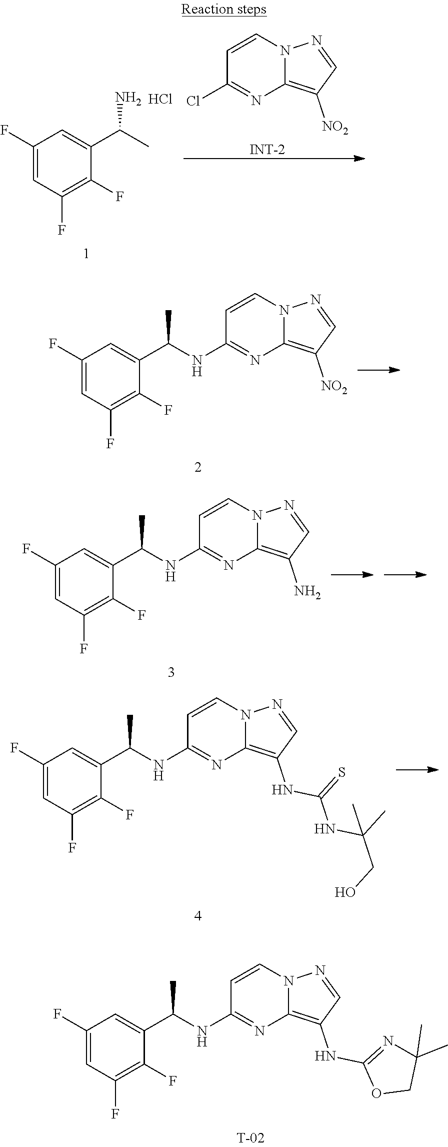

### (1) Synthesis of Compound 2

20 ml of anhydrous ethanol was added to 2.04 g of compound 1 (1.0 eq), followed by INT-2 (1.9 g, 1.0 eq) and DIPEA (6.34 ml, 3.0 eq), respectively. After replacing with nitrogen, the reaction was carried out at 60° C. for 4 h. The reaction was monitored by TLC until it was completed. Then ethanol was evaporated. Water was added to the reaction system, and EA (50 ml×3) was added for extraction. EA phases were combined, dried over anhydrous sodium sulfate, filtered, spin-dried and purified by column chromatography to obtain 1.75 g of compound 3.

### (2) Synthesis of Compound 3

Ethanol (40 ml) and water (60 ml) were added to compound 2 (1.75 g, 1.0 eq), then iron powder (0.87 g, 3.0 eq) and ammonium chloride (0.83 g, 3.0 eq) were added, heated to 85° C. to react for 2 h. The reaction was monitored by TLC until it was completed. Then ethanol was spin-dried. Water was added to the reaction system, and then EA (50 ml×3) was added for extraction. EA phases were collected, dried over anhydrous sodium sulfate, filtered and spin-dried to obtain 1.5 g of compound 3.

### (3) Synthesis of Compound 4

1,1′-Thiocarbonyldiimidazole (0.7 g, 1.1 eq) was dissolved in THF, then cooled to −10° C., and a solution of compound 3 (1.1 g, 1.0 eq) in THE was added under stirring. After stirring for 5 min, a solution of 2-amino-2-methyl-1-propanol (0.41 g, 1.3 eq) in THE was added, and naturally warmed to room temperature to react overnight. The reaction was monitored by TLC until it was completed. Water was added to the reaction system, and then EA (50 ml×3) was added for extraction. EA phases were collected, dried over anhydrous sodium sulfate, filtered, spin-dried, and purified by column chromatography to obtain 1.5 g of compound 4.

### (4) Example 2: Synthesis of Compound T-02

1.5 g of compound 4 (1.0 eq) was dissolved in THF, then TosCl(588 mg, 0.9 eq) and NaOH(820.8 mg, 6 eq) were added and stirred at room temperature overnight. The reaction was monitored by TLC until it was completed. Water was added to the reaction system, and then EA (30 ml×3) was added for extraction. The EA phases were combined, dried over anhydrous sodium sulfate, filtered, spin-dried and purified by column chromatography to obtain 0.7 g of T-02, [M+H]⁺=405.2.

### Example 3

Synthetic Route:

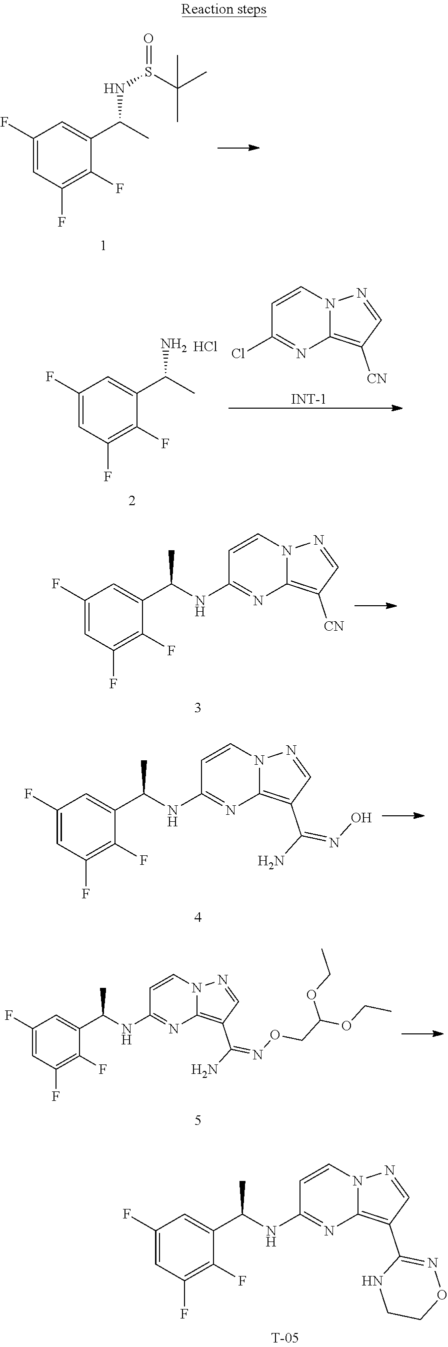

### (1) Synthesis of Compound 2

Dioxane hydrochloride (30 ml) was added to compound 1 (36.4 g, 1.0 eq), and reacted at room temperature for 2 h. The reaction was monitored by TLC until it was completed. The solvent was spin-dried, PE was added to slurry, filtered, and the filter cake was collected and spin-dried to obtain 26.2 g of compound 2.

### (2) Synthesis of Compound 3

Anhydrous ethanol was added to compound 2 (20 g, 1.0 eq), followed by INT-1 (16.8 g, 1.0 eq) and DIPEA (46.2 ml, 4.0 eq), respectively. After replacing with nitrogen, the reaction was carried out at 60° C. for 4 h. The reaction was monitored by TLC until it was completed. Then ethanol was spin-dried. Water was added to the reaction system and a solid was precipitated, filtered, spin-dried and purified by column chromatography to obtain 26.6 g of compound 3.

### (3) Synthesis of Compound 4

Anhydrous ethanol (100 ml) and 1,4-dioxane (100 ml) were added to compound 3 (10.6 g, 1.0 eq), then hydroxylamine hydrochloride (13.9 g, 6.0 eq) and potassium carbonate (55.4 g, 12.0 eq) were added, replaced with nitrogen gas, and reacted at 80° C. for 16 h. After the reaction monitored by TLC was completed, filtered, spin-dried and directly purified by column chromatography to obtain 8.3 g of compound 4.

### (4) Synthesis of Compound 5

Compound 4 (3.3 g, 1.0 eq) was dissolved in DMSO, then bromoacetal (3.7 g, 2.0 eq) and KOH(2.48 g, 4.0 eq) were added, and heated to 60° C. to react for 6 h. The reaction was monitored by TLC until it was completed. After water was added to the reaction system, a solid was precipitated out, EA(50 ml×3) was added for extraction, and EA phases were combined, dried over anhydrous sodium sulfate, filtered, spin-dried and purified by column chromatography to obtain 1.9 g of compound 5.

### (5) Example 3: Synthesis of Compound T-05

Compound 5 (1.9 g, 1.0 eq) was dissolved in DCE(20 ml), and then TFA(2 ml) was added to react at 80° C. for 1 h. TLC showed the raw materials disappeared, then sodium cyanoborohydride (1.02 g, 4.0 eq) was added in portions under cooling. After addition, the mixture was reacted at 80° C. overnight. After the reaction was completed, the reaction system was cooled and added with aqueous sodium bicarbonate solution to adjust PH to 7-8, followed by addition of DCM(50 ml×3) to extract. DCM phases were combined, dried over anhydrous sodium sulfate, filtered and spin-dried and purified by column chromatography to obtain 400 mg of T-05, [M+H]+=377.2.

### Example 4

Synthetic route:

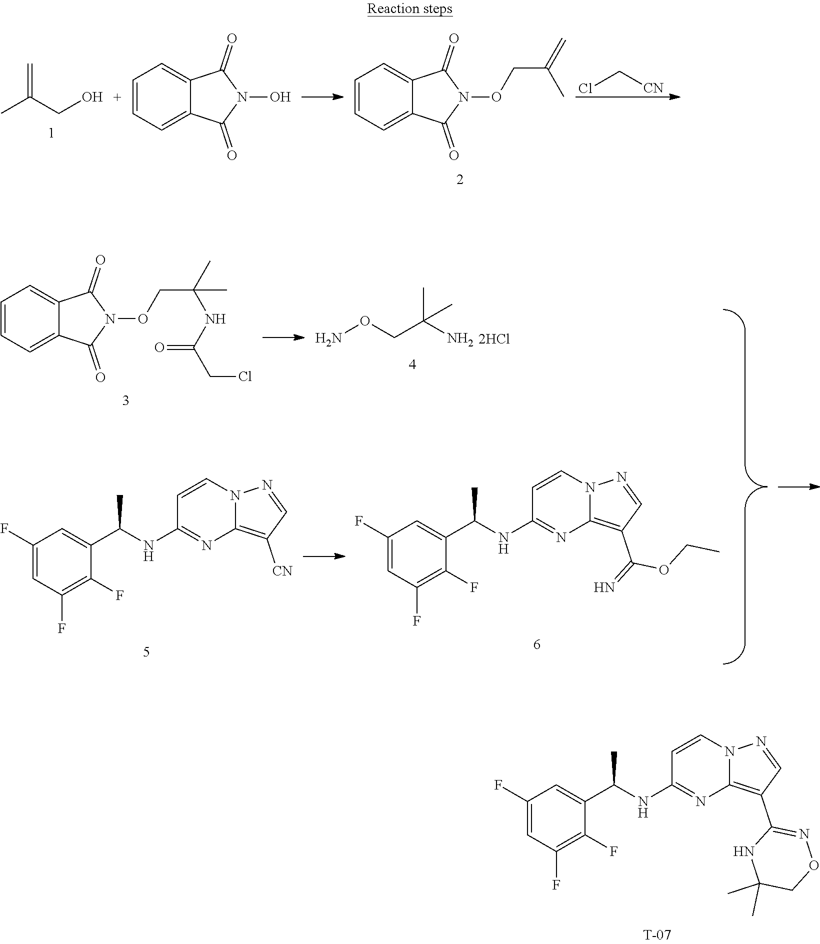

### (1) Synthesis of Compound 2

Under the protection of N2, compound 1 (2 g, 1 eq) was dissolved in 40 ml THE at 0° C., then N-hydroxyphthalimide (4.53 g, 1 eq) and PPh₃ (8.7 g, 1.2 eq) were added, then DEAD(5.8 g, 1.2 eq) was added dropwise to the reaction system, and gradually warmed to room temperature to react overnight. After the reaction was completed, the mixture was mixed with silica gel and purified by column chromatography to obtain 4.4 g of compound 2.

### (2) Synthesis of Compound 3

At 0° C., compound 2 (3.7 g, 1 eq) was dissolved in a mixed solvent of chloroacetonitrile (3.23 ml, 3 eq) and acetic acid (3 ml, 3 eq), then 98% H₂SO₄ (3.5 ml, 2 eq) was slowly added dropwise to the reaction system, the addition step was exothermic, and the reaction was carried out at room temperature for 1.5 h. After the reaction was completed, ice water and EA was slowly added for extraction, and the mixture was mixed with silica gel and purified by column chromatography to obtain 2 g of compound 3.

### (3) Synthesis of Compound 4

Compound 3 (2 g, 1 eq) was added into 6N HCl(40 ml), and the reaction was refluxed for 1.5 h. After the reaction was completed, the mixture was directly spin-dried, then dissolved in water, washed twice with MTBE, the aqueous phase was spin-dried, and slurried with EtOH to obtain 0.3 g of compound 4.

### (4) Synthesis of Compound 6

Compound 5 (0.3 g, 1 eq) was added to 6N hydrochloric acid ethanol (2 ml) and reacted overnight at room temperature. Then K₂CO₃ (0.7 g, 3 eq) was added, and the mixture was stirred for 1 hour, filtered and spin-dried to obtain 0.2 g of compound 5, which was used directly in the next step.

### (5) Example 4: Synthesis of Compound T-07

Compound 6 (0.2 g, 1 eq) was added to 2 ml acetic acid and then compound 4 (0.2 g, 3 eq) was added to react at 100° C. under the protection of N2. After the reaction was completed, aqueous NaHCO₃ was used to adjust to base and EA was used to extract and spin-dried to obtain 0.3 g of final compound T-07, [M+H]⁺=405.2.

### Example 5

Synthetic Route:

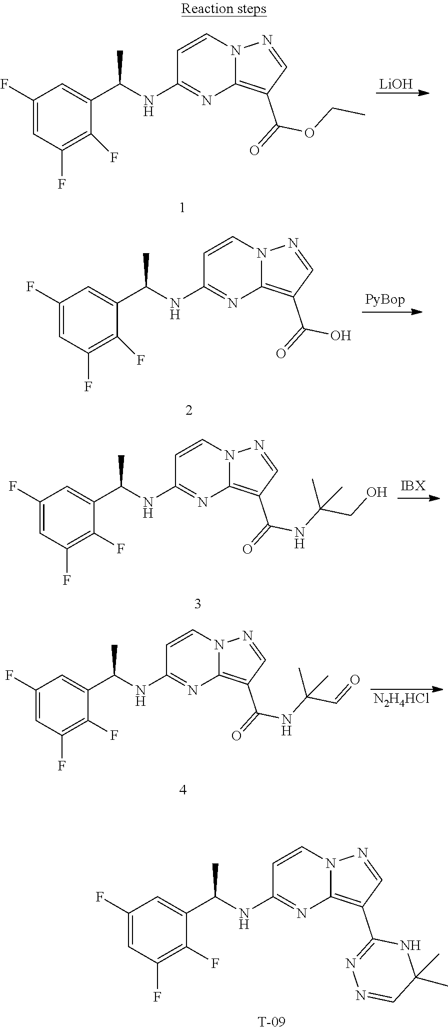

(1) Synthesis of compound 2: compound 1 (0.6 g, 1.65 mmol) was weighted into a 100 ml single-mouth flask, THF(6 ml), methanol(3 ml) and water(1 ml) were added and then lithium hydroxide (0.35 g, 8.25 mmol, 5 eq) was added and the mixture was stirred overnight at room temperature under the protection of nitrogen. The next day, the mixture was treated and purified by column chromatography to obtain 0.38 g of product with a yield of 68%.

(2) Synthesis of compound 3: compound 2 (1.425 g, 4.24 mmol) was weighed into a 250 ml single-mouth flask, 80 ml of dichloromethane was added, then 2-amino-2-methyl-1-propanol (1.34 g, 12.73 mmol, 3 eq), DIEA(1.095 g, 8.48 mmol, 2 eq), and PyBop(2.65 g, 5.09 mmol, 1.2 eq) were added under the protection of nitrogen. The mixture was stirred at room temperature for 4 hours. TLC showed that the reaction was completed. The reaction solution was treated and purified by column chromatography several times to obtain 1.61 g of white solid product.

(3) Synthesis of compound 4: compound 3 (0.284 g) and EA(30 ml) were added to a 100 ml single-mouth flask, IBX(0.39 g) was added and stirred at room temperature for 10 min, then heated to 85° C. to react for 4 hours under the protection of nitrogen. The reaction solution was treated and purified by column chromatography to obtain 0.255 g of white solid product.

### (4) Example 5: Synthesis of Compound T-09

Compound 4 (0.2 g) was added into a 100 ml single-mouth flask, then hydrazine monohydrochloride(33.8 mg, 1 eq), sodium hydroxide solid (19.76 mg, 1 eq) and 20 ml of anhydrous ethanol were added, stirred at room temperature for 20 min, then a drop of HOAc was added to react overnight at 80° C. TLC monitored, and the reaction solution was treated and purified by column chromatography to obtain 130 mg of product, [M+H]+=402.2.

### Example 6

Synthetic Route:

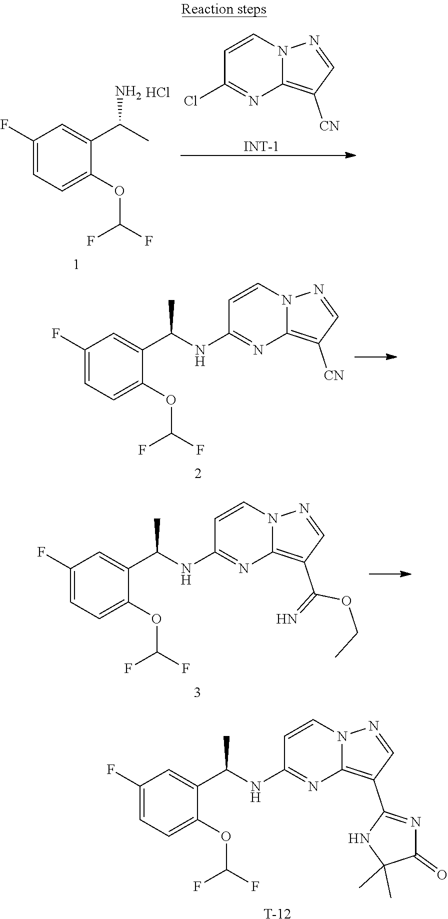

### (1) Synthesis of Compound 2

Compound 1 (1 g, 4.14 mmol, 1 eq) and INT-1 (0.75 g, 4.23 mmol, 1.02 eq) were added to ethanol, then triethylamine (1.25 g, 12.44 mmol, 3 eq) was added, and the mixture was raised to 55° C. to react for 2 hours. TLC monitored until the raw materials were reacted completely. The reaction solution was cooled, a large amount of water was added to the reaction system, and a solid was precipitated out, filtered, and the filter cake was washed with petroleum ether, and dried to obtain 1.2 g of compound 2.

### (2) Synthesis of Compound 3

Compound 2 (1.2 g, 3.45 mmol, 1 eq) was dissolved in EtOH/HCl(50 ml) and stirred at room temperature overnight. The reaction was monitored by TLC until it was completed. Ethanol was spin-dried, and then NaHCO₃ aqueous solution was added to the system to adjust PH value to 7-8, and EA(50 ml×3) was added for extraction. EA phases were combined, dried over anhydrous sodium sulfate, filtered, spin-dried and purified by column chromatography to obtain 0.75 g of compound 3.

### (3) Example 6: Synthesis of Compound T-12

Compound 3 (0.75 g, 1.90 mmol, 1 eq) was dissolved in xylene (10 ml), then ethyl 2-amino-isobutyrate (0.75 g, 5.72 mmol, 3 eq) and 2-3 drops of acetic acid(catalyst) were added. After replacing with nitrogen, the mixture was heated up to 130° C. to react for 6 h. The reaction was monitored by TLC until it was completed, and then NaHCO₃ aqueous solution was added to the system to adjust PH value to 7-8, and EA(50 ml×3) was added for extraction. EA phases were combined, dried over anhydrous sodium sulfate, filtered, spin-dried and purified by column chromatography to obtain 169 mg of T-12, [M+H]+=433.2.

### Example 7

Synthetic route:

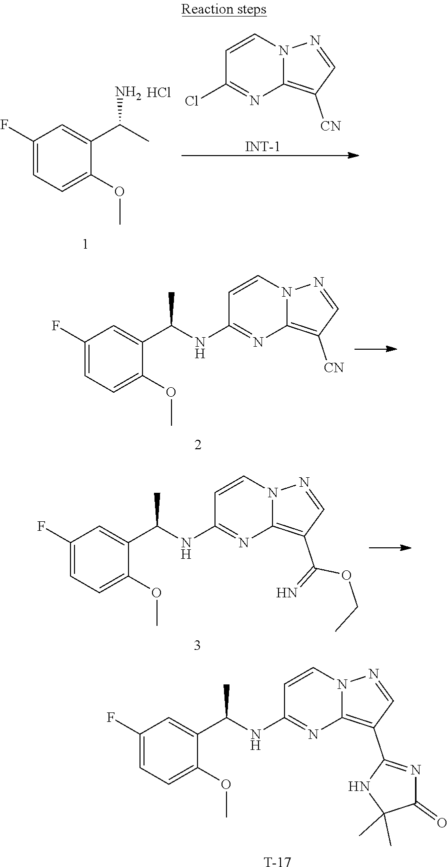

### (1) Synthesis of Compound 2

50 ml Anhydrous ethanol was added to 4.6 g of compound 1 (1.0 eq), followed by INT-1 (4.0 g, 1.0 eq) and DIPEA (11.7 ml, 4.0 eq), respectively. After replacing with nitrogen, the reaction was carried out at 60° C. for 4 h. The reaction was monitored by TLC until it was completed. Then ethanol was spin-dried. Water was added to the reaction system and a solid was precipitated out. The filter cake was collected and spin-dried to obtain 3.5 g of compound 2.

### (2) Synthesis of Compound 3

3.5 g of compound 2 (1.0 eq) was dissolved in EtOH/HCl(50 ml) and stirred at room temperature overnight. The reaction was monitored by TLC until it was completed. Ethanol was spin-dried, and then NaHCO₃ aqueous solution was added to system to adjust PH value to 7-8, EA(50 ml×3) was added for extraction. EA phases were combined, dried over anhydrous sodium sulfate, filtered, spin-dried and purified by column chromatography to obtain 3.7 g of compound 3.

### (3) Example 7: Synthesis of Compound T-17

Compound 3 (1.7 g, 1.0 eq) was dissolved in xylene (17 ml), then ethyl 2-amino-isobutyrate (1.87 g, 3 eq) and acetic acid (0.002 g, 0.006 eq) were added. After replacing with nitrogen, the mixture was heated up to 130° C. to react for 6 h. The reaction was monitored by TLC until it was completed, and then NaHCO₃ aqueous solution was added to the system to adjust PH value to 7-8, and EA(50 ml×3) was added for extraction. EA phases were combined, dried over anhydrous sodium sulfate, filtered, spin-dried and purified by column chromatography to obtain 320 mg of T-17, [M+H]⁺=397.2.

### Example 8

Synthetic Route:

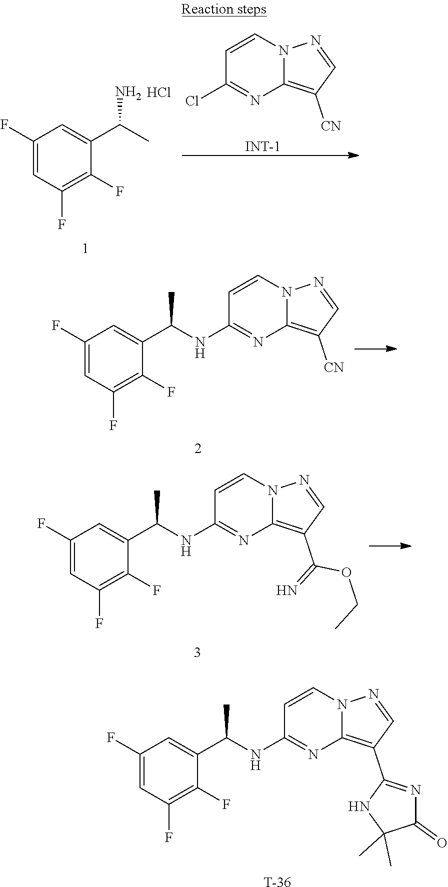

### (1) Synthesis of Compound 2

Compound 1 (5 g, 20.7 mmol, 1 eq) and INT-1 (3.75 g, 21.15 mmol, 1.02 eq) were added to ethanol, then triethylamine (6.25 g, 62.2 mmol, 3 eq) was added, and the mixture was raised to 55° C. to react for 2 hours. TLC monitored until the raw materials were reacted completely. The reaction solution was cooled, a large amount of water was added to the reaction system, and a solid was precipitated out, filtered, and the filter cake was washed with petroleum ether, and dried to obtain 5.6 g of compound 2.

### (2) Synthesis of Compound 3

Compound 2 (4 g, 12.6 mmol, 1 eq) was dissolved in EtOH/HCl(100 ml, poor solubility) and stirred at room temperature overnight. The reaction was monitored by TLC until it was completed. Ethanol was spin-dried, and then NaHCO₃ aqueous solution was added to system to adjust PH value to 7-8, EA(50 ml×3) was added for extraction. EA phases were combined, dried over anhydrous sodium sulfate, filtered, spin-dried and purified by column chromatography to obtain 2.4 g of compound 3.

### (3) Example 8: Synthesis of Compound T-36

Compound 3 (1.2 g, 3.3 mmol, 1 eq) was dissolved in xylene (20 ml), then ethyl 2-amino-isobutyrate (1.29 g, 9.91 mmol, 3 eq) and 0.1 mL of acetic acid(catalyst) were added. After replacing with nitrogen, the mixture was heated up to 130° C. to react for 6 h. The reaction was monitored by TLC until it was completed, and then NaHCO₃ aqueous solution was added to the system to adjust PH value to 7-8, and EA(50 ml×3) was added for extraction. EA phases were combined, dried over anhydrous sodium sulfate, filtered, spin-dried and purified by column chromatography to obtain 520 mg of T-36, [M+H]+=403.1.

### Example 9

Synthetic Route:

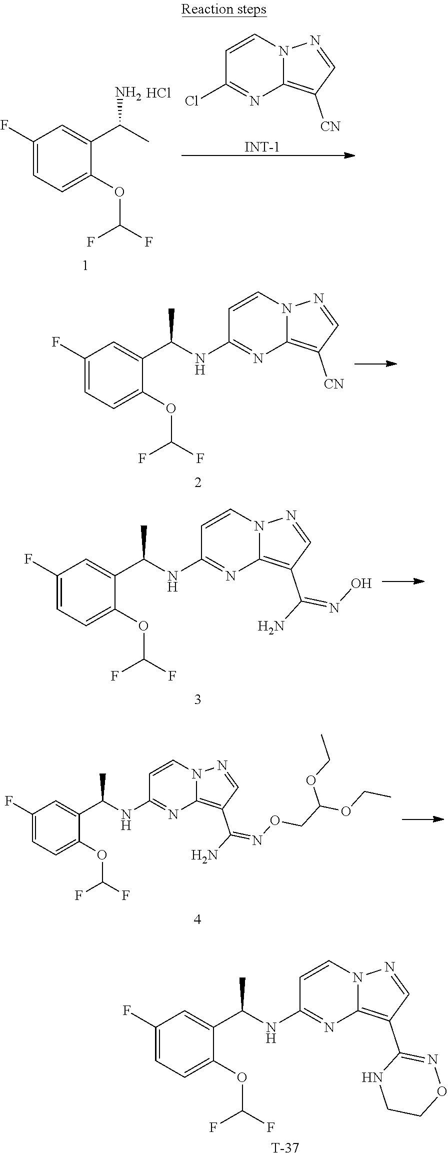

### (1) Synthesis of Compound 2

Compound 1 (1 g, 4.14 mmol, 1 eq) and INT-1 (0.75 g, 4.23 mmol, 1.02 eq) were added to ethanol, then triethylamine (1.25 g, 12.44 mmol, 3 eq) was added, and raised to 55° C. to react for 2 hours. TLC monitored until the raw materials were reacted completely. The reaction solution was cooled, a large amount of water was added to the reaction system, and a solid was precipitated out, filtered, and the filter cake was washed with petroleum ether, and dried to obtain 1.2 g of compound 2.

### (2) Synthesis of Compound 3

Compound 2 (1.2 g, 3.45 mmol, 1 eq) was added to anhydrous ethanol (10 ml) and 1,4-dioxane (10 ml), then hydroxylamine hydrochloride (1.9 g, 27.6 mmol, 8 eq) and potassium carbonate (3.8 g, 27.6 mmol, 8 eq) were added. After replacing with nitrogen, the mixture was heated up to 80° C. to react for 16 h. The reaction was monitored by TLC until it was completed. The reaction solution was filtered, spin-dried and directly purified by column chromatography to obtain 670 mg of compound 3.

### (3) Synthesis of Compound 4

Compound 3 (0.67 g, 1.76 mmol, 1 eq) was dissolved in DMSO, then bromoacetal (0.69 g, 3.52 mmol, 2 eq) and KOH(0.39 g, 7.05 mmol, 4 eq) were added, and the mixture was heated to 60° C. to react for 6 h. The reaction was monitored by TLC until it was completed. Water was added to the reaction system, a solid was precipitated out, EA(10 ml×3) was added for extraction, and EA phases were combined, dried over anhydrous sodium sulfate, filtered, spin-dried and purified by column chromatography to obtain 0.48 g of compound 4.

### Example 9: Synthesis of Compound T-37

Compound 4 (0.48 g, 0.96 mmol, 1 eq) was dissolved in DCE(5 ml), and then TFA(0.2 ml) was added to react at 80° C. for 1 h. TLC showed the raw materials were disappeared, then sodium cyanoborohydride (0.24 g, 3.87 mmol, 4 eq) was added in portions under cooling. After addition, the mixture was reacted at 80° C. overnight. After the reaction was completed, the reaction system was cooled and added with aqueous sodium bicarbonate solution to adjust PH to 7-8, followed by adding DCM(50 ml×3) for extraction. DCM phases were combined, dried over anhydrous sodium sulfate, filtered and spin-dried and purified by column chromatography to obtain 90 mg of T-37, [M+H]+=407.1.

### Example 10

Synthetic Route:

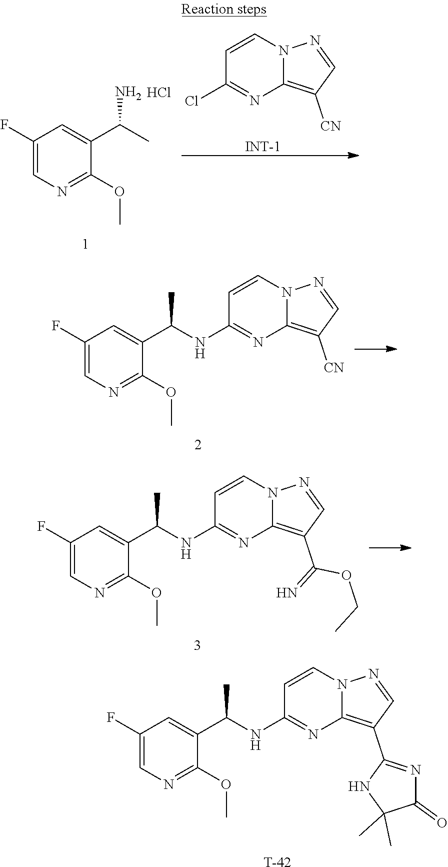

### (1) Synthesis of Compound 2

20 ml Anhydrous ethanol was added to 1.85 g of compound 1 (1.0 eq), followed by INT-1 (1.6 g, 1.0 eq) and DIPEA (4.68 ml, 4.0 eq), respectively. After replacing with nitrogen, the reaction was carried out at 60° C. for 4 h. The reaction was monitored by TLC until it was completed. Then ethanol was spin-dried. Water was added to the reaction system and a solid was precipitated out. The filter cake was collected and spin-dried to obtain 1.12 g of compound 2.

### (2) Synthesis of Compound 3

Compound 2 (1.12 g, 1.0 eq) was dissolved in EtOH/HCl(20 ml) and stirred at room temperature overnight. The reaction was monitored by TLC until it was completed. Ethanol was spin-dried, and then NaHCO₃ aqueous solution was added to system to adjust PH value to 7-8, EA(50 ml×3) was added for extraction. EA phases were combined, dried over anhydrous sodium sulfate, filtered, spin-dried and purified by column chromatography to obtain 1.5 g of compound 3.

### (3) Example 10: Synthesis of Compound T-42

Compound 3 (1.5 g) was dissolved in xylene (15 ml), then ethyl 2-amino-isobutyrate (0.65 g, 3.0 eq) and acetic acid (1.85 mg, 0.006 eq) were added. After replacing with nitrogen, the mixture was heated up to 130° C. to react for 6 h. The reaction was monitored by TLC until it was completed. The reaction solution was cooled down, and then NaHCO₃ aqueous solution was added to the system to adjust PH value to 7-8, and EA(50 ml×3) was added for extraction. EA phases were combined, dried over anhydrous sodium sulfate, filtered, spin-dried and purified by column chromatography to obtain 680 mg of T-42, [M+H]+=398.2.

### Example 11

Synthetic Route:

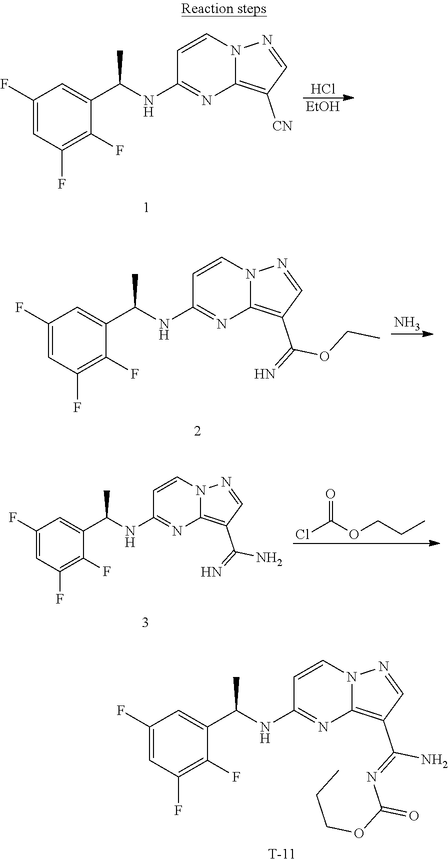

### (1) Synthesis of Compound 2

Compound 1 (2 g, 12.6 mmol, 1 eq) was dissolved in EtOH/HCl(50 ml, poor solubility) and stirred at room temperature overnight. The reaction was monitored by TLC until it was completed. Ethanol was spin-dried, and then NaHCO₃ aqueous solution was added to system to adjust PH value to 7-8, EA(50 ml×3) was added for extraction. EA phases were combined, dried over anhydrous sodium sulfate, filtered, spin-dried and purified by column chromatography to obtain 1.3 g of compound 2.

### (2) Synthesis of Compound 3

Compound 2 (1.3 g) was added to a solution (50 mL) of ammonia gas in ethanol, and heated to 50° C. to react under stirring overnight. The reaction was directly spin-dried and purified by column chromatography to obtain 0.8 g of compound 3, [M+H]⁺=335.1.

(3) Compound 3 (0.8 g, 2.4 mmol) was added to 50 mL of dichloromethane, then triethylamine (3.0 eq, 7.2 mmol) was added, and n-propyl chloroformate (1.2 eq, 2.9 mmol) was added dropwise at 0° C. After addition, the reaction solution was slowly warmed to room temperature overnight, washed with saturated brine, then dried, spin-dried and purified by column chromatography to obtain 325 mg of final product T-11, [M+H]⁺=421.1.

Meanwhile, with reference to the above examples, examples 12-28 were synthesized, as detailed in Table 1:

TABLE 1

| Example | Structural formula of compound | Characterization data (MS) of compound |
|---|---|---|
| 12 | 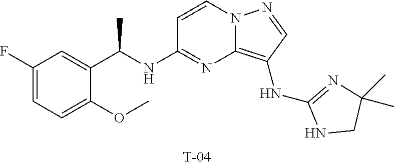 [SMILES: COc1ccc(F)cc1[C@@H](C)Nc1ccn2ncc(NC3=NC(C)(C)CN3)c2n1] | [M + H]⁺ = 398.2 |
| 13 | 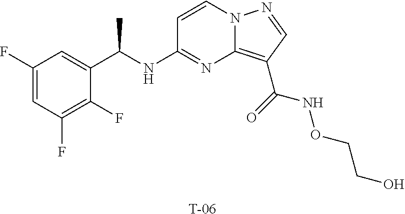 [SMILES: C[C@@H](Nc1ccn2ncc(C(=O)NOCCO)c2n1)c1cc(F)cc(F)c1F ; "T-06"] | [M + H]⁺ = 396.1 |
| 14 | 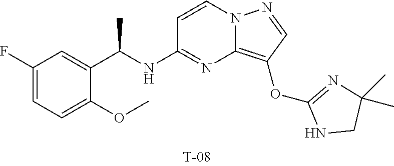 [SMILES: COc1ccc(F)cc1[C@@H](C)Nc1ccn2ncc(OC3=NC(C)(C)CN3)c2n1] | [M + H]⁺ = 399.2 |
| 15 | 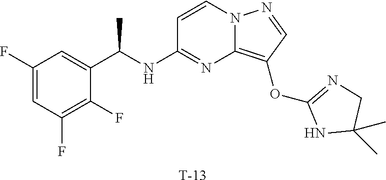 | [M + H]⁺ = 405.1 |
| 16 | 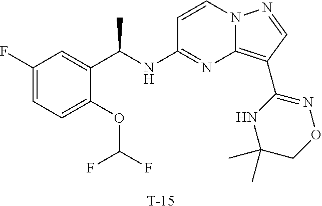 | [M + H]⁺ = 435.2 |
| 17 | 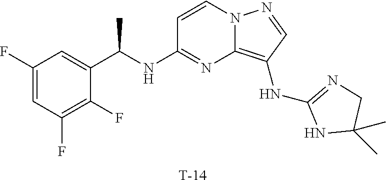 | [M + H]⁺ = 404.2 |
| 18 | 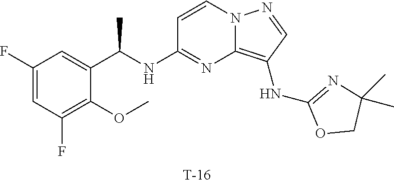 | [M + H]⁺ = 417.2 |
| 19 | 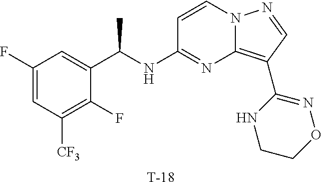 | [M + H]⁺ = 427.1 |
| 20 | 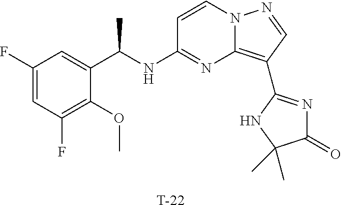 | [M + H]⁺ = 415.2 |
| 21 | 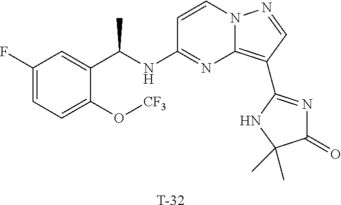 | [M + H]⁺ = 451.2 |
| 22 | 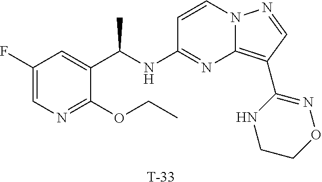 | [M + H]⁺ = 386.2 |
| 23 | 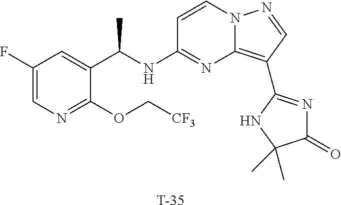 | [M + H]⁺ = 466.1 |
| 24 | 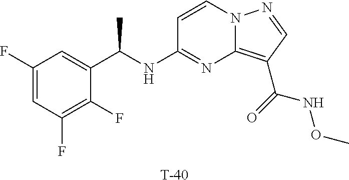 | [M + H]⁺ = 366.1 |
| 25 | 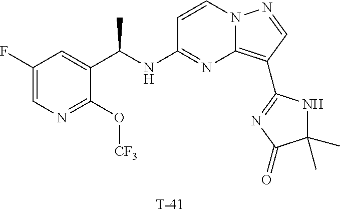 | [M + H]⁺ = 452.1 |
| 26 | 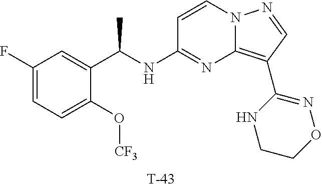 | [M + H]⁺ = 425.1 |
| 27 | 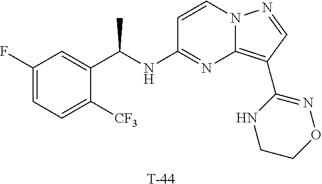 | [M + H]⁺ = 409.1 |
| 28 | 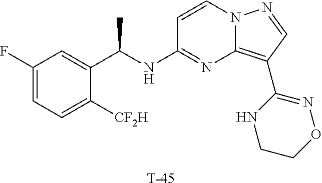 | [M + H]⁺ = 391.1 |

### Biological Activity Test Example

### Test Example 1: Inhibitory Activity of the Compounds of the Invention Against ROS1, NTRK and ALK and their Drug-Resistant Kinases

Inhibition of protein kinase activity by compounds was carried out on the Radio-tagged HotSpot kinase experimental platform of Reaction Biology Corporation. Fresh reaction solution (20 mM TIEPESpH 7.5, 10 mM MgCl2, 1 mM EGTA, 0.02% Brij35, 0.02 mg/ml BSA, 0.1 mM Na3VO4, 2 mM DTT, 100 DMS0) containing corresponding substrates was prepared, cofactor and kinase to be tested were added into the above solution and mixed gently. Echo550 pipetting system was used to add the test compound DMSO solution to each well (the blank control group was added with the corresponding volume of DMSO), then 33P-ATP (with a final specific activity of 0.01 μCi/μL) was added to start the reaction. The reaction solution was incubated at room temperature for 120 minutes. Transferred the incubated reaction solution to P81 ion exchange chromatographic paper (Whatman #3698-915), eluted with 0.7500 phosphoric acid solution, and the amount of radioactive phosphorylated substrate remaining on the chromatographic paper was detected.

Table 2 showed the inhibitory activity IC₅₀ Value of the compounds of the present invention against ROS1, NTRK and ALK and the drug-resistant kinases thereof, A<0.5 nM, 0.5 nm≤B≤5.0 nM, 5.0 nM K C K 50 nM, 50 nM≤D≤500 nM, E>500 nM.

TABLE 2

| | | ROS1 | | TRKA | | ALK |
|---|---|---|---|---|---|---|
| | ROS1 | (G2032R) | TRKA | (G677C) | ALK | (G1202R) |
| Example | (IC₅₀/nM) | (IC₅₀/nM) | (IC₅₀/nM) | (IC₅₀/nM) | (IC₅₀/nM) | (IC₅₀/nM) |
| 1 | B | B | B | B | B | C |
| 2 | B | B | B | B | B | C |
| 3 | A | A | A | A | B | B |
| 4 | A | B | A | B | B | C |
| 5 | B | C | B | C | C | C |
| 6 | A | A | A | A | B | C |
| 7 | A | A | A | A | B | C |
| 8 | A | A | A | A | B | C |
| 9 | A | A | A | A | B | C |
| 10 | A | A | A | A | B | C |
| 11 | B | C | B | C | D | D |
| 12 | B | C | B | C | C | C |
| 13 | C | D | C | D | D | D |
| 14 | B | C | B | C | D | D |
| 15 | B | C | B | C | D | D |
| 16 | B | C | B | C | C | D |
| 17 | B | C | B | C | D | D |
| 18 | B | C | B | C | C | D |
| 19 | B | C | B | C | C | D |
| 20 | A | B | A | B | B | C |
| 21 | A | B | A | B | B | C |
| 22 | A | B | A | B | B | C |
| 23 | A | B | A | B | C | D |
| 24 | C | D | C | D | C | D |
| 25 | A | B | A | B | B | C |
| 26 | A | B | A | B | B | C |
| 27 | B | D | B | D | C | D |
| 28 | B | D | B | D | C | D |
| Staurosporine | 0.246 | 13.0 | 2.11 | | | |

The kinase activity test shows that the series compounds of the present invention have good inhibitory activity on ROS1, NTRK and ALK and the drug-resistant mutations thereof, especially the inhibitory activity on drug-resistant mutations is better.

The compounds of the present invention have better inhibitory activity against one or more of ROS1, NTRK and ALK and the drug-resistant mutations thereof than currently clinically available drug LOXO-101.

Most of the compounds of the invention have better or equivalent activity against one or more of ROS1, NTRK and ALK and the drug-resistant mutations thereof than LOXO-195 and TPX-0005.

The compounds of the invention have great potential for use in the treatment of diseases mediated by ROS1, NTRK, ALK and the like.

### Test Example 2: Inhibition of Cell Proliferation by Compounds

The experiment of inhibiting cell proliferation by compounds was carried out in Hefei Zhongkeprecedo Biomedical Technology Co., Ltd. The Ba/F3 engineered cell line stably transfected with different kinase genes was recovered with RPMI 1640 medium (Biological Industries, Israel)+10% fetal bovine serum (Biological Industries, Israel)+1% double antibody (Penicillin Streptomycin solution, Coring, USA) and cultivated two generations. The logarithmic growth phase cell suspension was taken, and 2000 cells/well were inoculated on 96-well white cell culture plate (Corning 3917, NY, USA) with a volume of 95 μL per well. 5 μL of 20×DMSO solution of the compound to be tested was added into the culture plate containing 95 μL of cell suspension. The blank control group was added with corresponding volume of DMSO, mixed well, and incubated in a 5% CO₂ incubator at 37° C. for 72 hours. CellTiter-Glo was used to detect cell viability.

Table 3 showed the inhibitory activity IC₅₀ Value of the compounds of the present invention against ROS1, NTRK and ALK or their drug-resistant mutant Ba/F3 engineered cell lines.

TABLE 3

| | Ba/F3- | Ba/F3- | Ba/F3- |
|---|---|---|---|
| | CD74-ROS1- | LMNA-NTRK1- | TEL-ALK- |
| Example | G2032R(IC₅₀/nM) | G595R (IC₅₀/nM) | G1202R (IC₅₀/nM) |
| 6 | 24.1 | 13.4 | 110.3 |
| 7 | 54.6 | 36.1 | |
| 9 | 31.6 | 6.1 | 75.6 |
| 26 | 35.2 | 11.2 | 100.2 |

The cell activity test shows that the series compounds of the present invention have good inhibitory activity against ROS1, NTRK and ALK and their drug-resistant mutant Ba/F3 engineered cell lines, especially the inhibitory activity against drug-resistant mutations is better. The compounds of the invention have good inhibitory activity against ROS1, NTRK and ALK and their drug-resistant mutant BA/F3 engineering cell lines, and most of the compounds of the invention have excellent activity against ROS1, NTRK and ALK and their drug-resistant mutant BA/F3 engineering cell lines, and they have great potential to be applied to the treatment of diseases mediated by ROS1, NTRK and ALK and the like.

### Test Example 3 In Vivo Efficacy Study

Experimental Design

Cell culture: Ba/F3-CD74-ROS1-G2032R cell line was cultured with 1640 medium (Biological Industries)+10% fetal bovine serum (BI)+1% double antibody (Penicillin Streptomycin solution, Corning, USA) at 37° C. with 5% CO₂. Two passages were performed for one week. When the cell saturation was 80%-90% and the number reached the required level, the cells were collected, counted and inoculated.

Animals: BALB/c nude mice, female, 6-8 weeks of age, weighing 15-20 g. A total of 6 mice were required, provided by Beijing Vital River Laboratory Animal Technology Co., Ltd.

Tumor inoculation: 0.2 ml (2×10⁶) Ba/F3-CD74-ROS1-G2032R cells (plus stromal gel, volume ratio 1:1) were inoculated subcutaneously on the right posterior dorsum of each mouse, and group dosing was started when the average tumor volume reached about 150-200 mm³.

Animal Grouping and Dosing Regimen

The experimental groups and dosing regimen are shown in Table 4

TABLE 4

| | | | | Dosing | | |
|---|---|---|---|---|---|---|
| | | Dosing Group | | volume | | |
| Group | | and Dosage | | Parameter | | Frequency |
| Number | Animal | (mg/kg) | | (μl/g) | | of dosing |
| 1 | 3 | Vehicle | — | 10 | P.O. | BID × 2 weeks |
| 2 | 3 | Example 4 | 20 | 10 | P.O. | BID × 2 weeks |

Animal husbandry: After arrival, the animals should be kept in quarantine in the experimental environment for 7 days before starting the experiment.

Experimental index: Tumor volume and body weight were measured twice a week. Tumor volume was measured by vernier calipers with the formula TV=0.5 a×b2, wherein a is the long diameter of the tumor and b is the short diameter of the tumor.

The experimental results are shown in Table 5.

TABLE 5

| | Tumor Volume(mm³) | | | | |
|---|---|---|---|---|---|
| Tested Compound | 0 days | 4 days | 7 days | 11 days | 14 days |
| Blank control | 168.26 | 397.95 | 581.39 | 956.12 | 2343.39 |
| Example 4 | 167.31 | 216.42 | 292.24 | 375.67 | 688.91 |

As can be seen from Table 5: the compounds of the present invention have excellent tumor inhibitory activity and have good prospects for clinical application and drug.

All literatures mentioned in the present application are incorporated by reference herein, as though individually incorporated by reference. Additionally, it should be understood that after reading the above teaching, many variations and modifications may be made by the skilled in the art, and these equivalents also fall within the scope as defined by the appended claims.

1. A compound of formula I, or a stereoisomer, a tautomer, a crystalline form, a pharmaceutically acceptable salt, a hydrate, a solvate or a prodrug thereof, 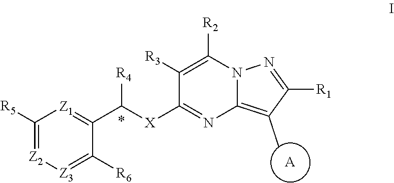
wherein,
is R or S configuration;
Z₁, Z₂ and Z₃ are each independently selected from: N or CR₁₃;
X is selected from the group consisting of NR₆, O, CR₁R₂, S, S(O) and S(O)₂;
R₁, R₂, R₃, R₄, R₅, R₆ and R₁₃ are each independently selected from the substituted or unsubstituted group consisting of H, halogen, amino, cyano, nitro, hydroxyl, acyl, ester group, C1-C6 alkyl, C3-C8 cycloalkyl, 3-8 membered heterocyclyl, C1-C6 alkoxy, C6-C14 aryl and 5-14 membered heteroaryl; wherein the substituted means being substituted by one or more R;
A is selected from the group consisting of 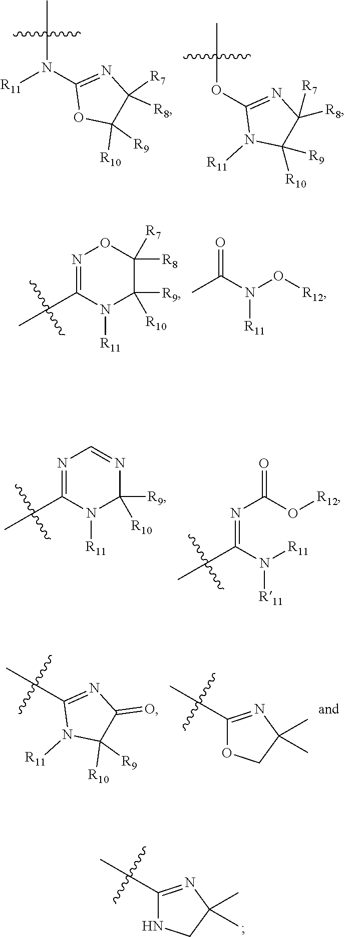
R₇, R₈, R₉, R₁₀, R₁₁ and R′₁₁ are each independently selected from the substituted or unsubstituted group consisting of hydrogen atom, cyano, C1-C6 alkyl, C3-C8 cycloalkyl, C6-C14 aryl and 5-14 membered heteroaryl; wherein the substituted means being substituted by one or more R;
R₁₂ is selected from the group consisting of C1-C6 alkyl and hydroxy-substituted C1-C6 alkyl;
R is selected from the group consisting of deuterium, halogen, amino, cyano, nitro, hydroxy, acyl, ester group, C1-C6 alkyl, C1-C6 haloalkyl, C3-C8 cycloalkyl, C3-C8 halocycloalkyl, C1-C6 alkoxy, C1-C6 haloalkoxy, C6-C14 aryl and 5-14 membered heteroaryl.

2. The compound of formula I, or a stereoisomer, a tautomer, a crystalline form, a pharmaceutically acceptable salt, a hydrate, a solvate or a prodrug thereof of claim 1, wherein it has a structure shown in formula II: 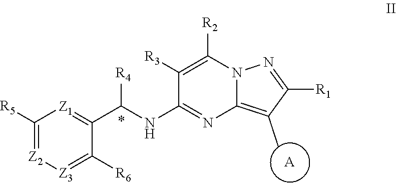
wherein,
is R or S configuration;
Z₁, Z₂, Z₃, R₁, R₂, R₃, R₄, R₅, R₆ and A are as defined in claim 1.

3. The compound of formula I, or a stereoisomer, a tautomer, a crystalline form, a pharmaceutically acceptable salt, a hydrate, solvate or a prodrug thereof of claim 1, wherein it has a structure shown in formula III or IV: 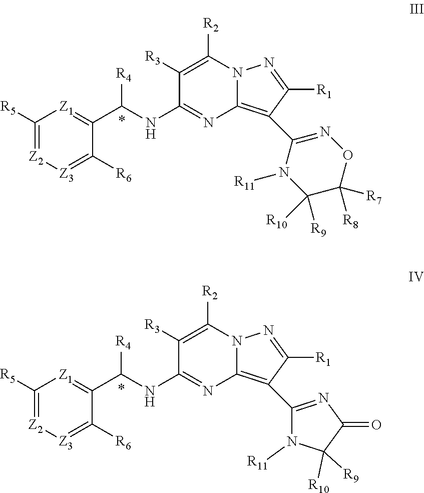
wherein,
is R or S configuration;
Z₁, Z₂, Z₃, R₁, R₂, R₃, R₄, R₅, R₆, R₇, R₈, R₉, R₁₀ and R₁₁ are as defined in claim 1.

4. The compound of formula I, or a stereoisomer, a tautomer, a crystalline form, a pharmaceutically acceptable salt, a hydrate, a solvate or a prodrug thereof of claim 1, wherein it has a structure shown in formula V or VI: 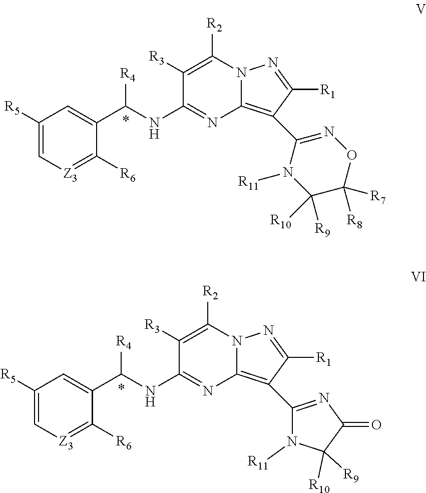
wherein,
is R or S configuration;
R₄ is selected from the group consisting of H, C1-C6 alkyl, C3-C6 cycloalkyl and C1-C6 haloalkyl;
Z₃, R₁, R₂, R₃, R₅, R₆, R₇, R₈, R₉, R₁₀ and R₁ are as defined in claim 1.

5. The compound of formula I, or a stereoisomer, a tautomer, a crystalline form, a pharmaceutically acceptable salt, a hydrate, a solvate or a prodrug thereof of claim 1, wherein R₁, R₂ and R₃ are each independently selected from the group consisting of hydrogen, halogen and amino;
R₄ is selected from the group consisting of hydrogen, C1-C6 alkyl, C3-C6 cycloalkyl and C1-C6 haloalkyl;
R₅ is selected from the group consisting of hydrogen and halogen;
R₆ is selected from the group consisting of hydrogen, halogen, C1-C6 alkyl, C3-C6 cycloalkyl, C1-C6 alkoxy, C1-C6 alkylamino, C1-C6 haloalkyl, C1-C6 haloalkoxy, C1-C6 haloalkylamino and C3-C6 halocycloalkyl;
R₇, R₈, R₉, R₁₀ and R₁₁ are each independently selected from the group consisting of hydrogen and substituted or unsubstituted C1-C6 alkyl; wherein, the “substituted” refers to being substituted by one or more groups selected from the group consisting of deuterium, halogen, amino, cyano, hydroxyl, acyl, ester group, C1-C6 alkyl, C1-C6 haloalkyl, C3-C6 cycloalkyl, C3-C6 halocycloalkyl, C1-C6 alkoxy, C1-C6 haloalkoxy, C6-C10 aryl and 5-10 membered heteroaryl.

6. The compound of formula I, or a stereoisomer, a tautomer, a crystalline form, a pharmaceutically acceptable salt, a hydrate, a solvate or a prodrug thereof of claim 1, wherein it has a structure shown in formula VII or VIII: 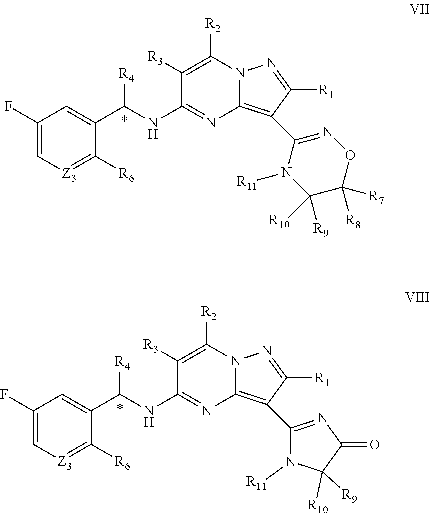
wherein
is R or S configuration;
Z₃, R₁, R₂, R₃, R₄, R₆, R₇, R₈, R₉, R₁₀ and Ru are as defined in claim 1.

7. The compound of formula I, or a stereoisomer, a tautomer, a crystalline form, a pharmaceutically acceptable salt, a hydrate, a solvate or a prodrug thereof of claim 1, wherein 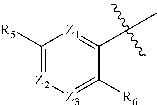
a moiety is selected from the substituted or unsubstituted group consisting of phenyl and pyridyl;
wherein, the “substituted” refers to being substituted by one or more groups selected from the group consisting of halogen, cyano, nitro, hydroxyl, C1-C6 alkyl, C1-C6 haloalkyl, C1-C6 alkoxy and C1-C6 haloalkoxy.

8. The compound of formula I, or a stereoisomer, a tautomer, a crystalline form, a pharmaceutically acceptable salt, a hydrate, a solvate or a prodrug thereof of claim 1, wherein R₅ is F.

9. The compound of formula I, or a stereoisomer, a tautomer, a crystalline form, a pharmaceutically acceptable salt, a hydrate, a solvate or a prodrug thereof of claim 1, wherein R₇, R₈, R₉ and R₁₀ are each independently selected from the group consisting of hydrogen, C1-C3 alkyl and C1-C3 haloalkyl.

10. The compound of formula I, or a stereoisomer, a tautomer, a crystalline form, a pharmaceutically acceptable salt, a hydrate, a solvate or a prodrug thereof of claim 1, wherein R₆ is selected from the group consisting of halogen, C1-C3 haloalkoxy and C1-C6 haloalkylamino.

11. The compound of formula I, a stereoisomer, a tautomer, a crystalline form, a pharmaceutically acceptable salt, a hydrate, a solvate or a prodrug of claim 1, wherein the compound is selected from the group consisting of

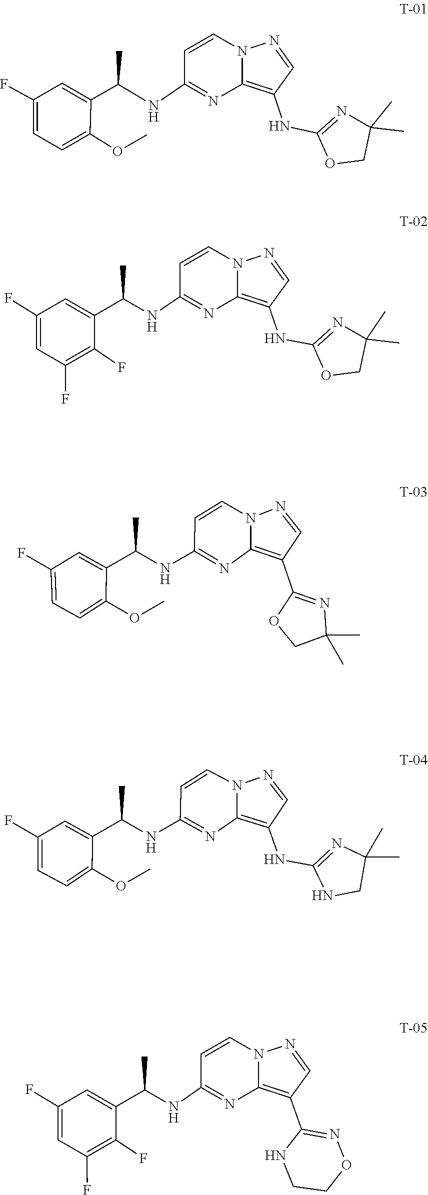 [SMILES: COc1ccc(F)cc1[C@@H](C)Nc1ccn2ncc(NC3=NC(C)(C)CO3)c2n1 ; C[C@@H](Nc1ccn2ncc(NC3=NC(C)(C)CO3)c2n1)c1cc(F)cc(F)c1F ; COc1ccc(F)cc1[C@@H](C)Nc1ccn2ncc(C3=NC(C)(C)CO3)c2n1 ; COc1ccc(F)cc1[C@@H](C)Nc1ccn2ncc(NC3=NC(C)(C)CN3)c2n1 ; C[C@@H](Nc1ccn2ncc(C3=NOCCN3)c2n1)c1cc(F)cc(F)c1F]

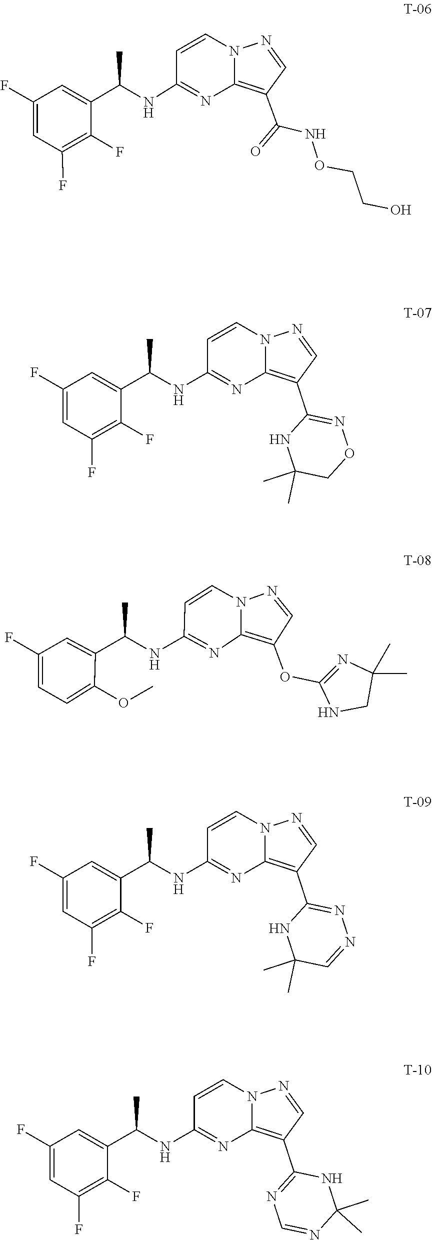 [SMILES: C[C@@H](Nc1ccn2ncc(C(=O)NOCCO)c2n1)c1cc(F)cc(F)c1F ; C[C@@H](Nc1ccn2ncc(C3=NOCC(C)(C)N3)c2n1)c1cc(F)cc(F)c1F ; COc1ccc(F)cc1[C@@H](C)Nc1ccn2ncc(OC3=NC(C)(C)CN3)c2n1 ; C[C@@H](Nc1ccn2ncc(C3=NN=CC(C)(C)N3)c2n1)c1cc(F)cc(F)c1F ; C[C@@H](Nc1ccn2ncc(C3=NC=NC(C)(C)N3)c2n1)c1cc(F)cc(F)c1F]

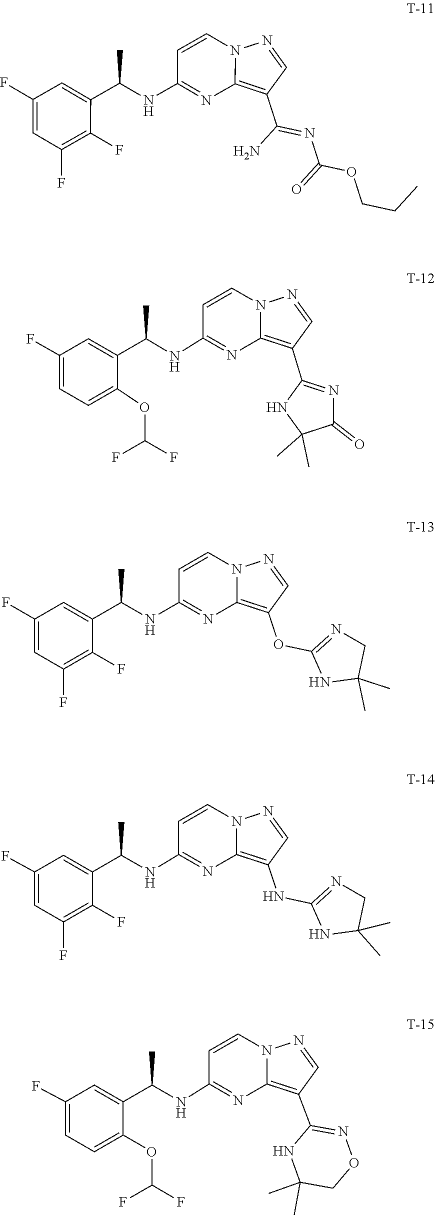 [SMILES: CCCOC(=O)/N=C(\N)c1cnn2ccc(N[C@H](C)c3cc(F)cc(F)c3F)nc12 ; C[C@@H](Nc1ccn2ncc(C3=NC(=O)C(C)(C)N3)c2n1)c1cc(F)ccc1OC(F)F ; C[C@@H](Nc1ccn2ncc(OC3=NCC(C)(C)N3)c2n1)c1cc(F)cc(F)c1F ; C[C@@H](Nc1ccn2ncc(NC3=NCC(C)(C)N3)c2n1)c1cc(F)cc(F)c1F ; C[C@@H](Nc1ccn2ncc(C3=NOCC(C)(C)N3)c2n1)c1cc(F)ccc1OC(F)F]

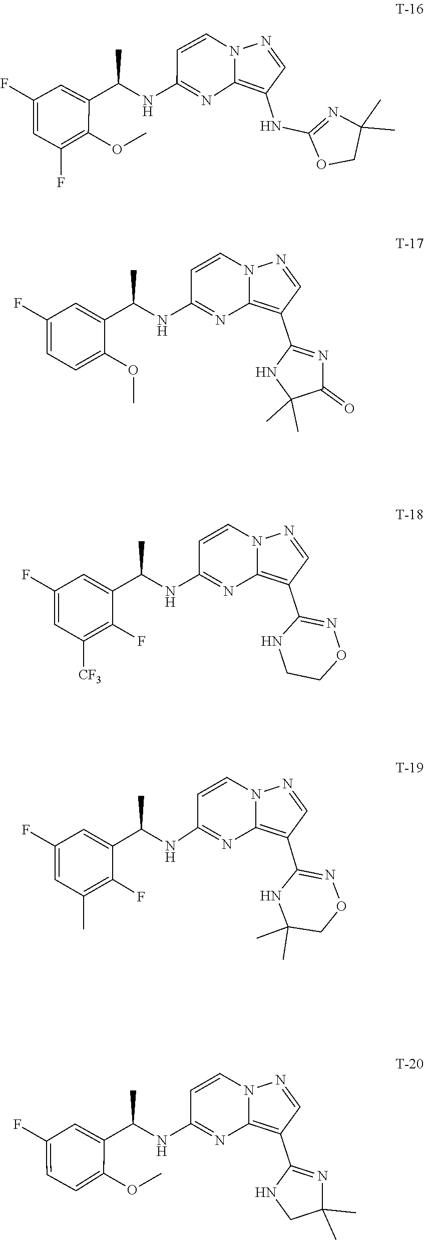 [SMILES: COc1c(F)cc(F)cc1[C@@H](C)Nc1ccn2ncc(NC3=NC(C)(C)CO3)c2n1 ; COc1ccc(F)cc1[C@@H](C)Nc1ccn2ncc(C3=NC(=O)C(C)(C)N3)c2n1 ; C[C@@H](Nc1ccn2ncc(C3=NOCCN3)c2n1)c1cc(F)cc(C(F)(F)F)c1F ; Cc1cc(F)cc([C@@H](C)Nc2ccn3ncc(C4=NOCC(C)(C)N4)c3n2)c1F ; COc1ccc(F)cc1[C@@H](C)Nc1ccn2ncc(C3=NC(C)(C)CN3)c2n1]

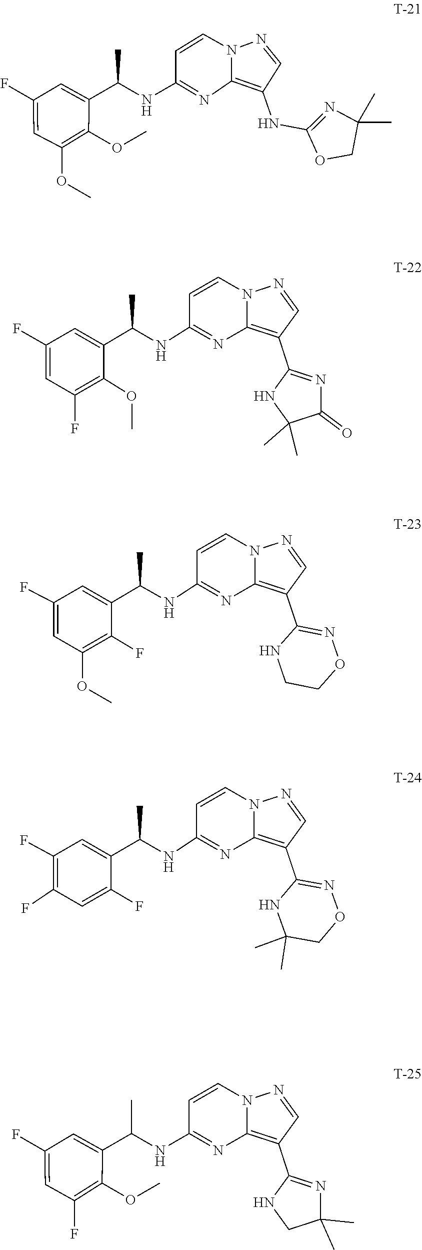 [SMILES: COc1cc(F)cc([C@@H](C)Nc2ccn3ncc(NC4=NC(C)(C)CO4)c3n2)c1OC ; COc1c(F)cc(F)cc1[C@@H](C)Nc1ccn2ncc(C3=NC(=O)C(C)(C)N3)c2n1 ; COc1cc(F)cc([C@@H](C)Nc2ccn3ncc(C4=NOCCN4)c3n2)c1F ; C[C@@H](Nc1ccn2ncc(C3=NOCC(C)(C)N3)c2n1)c1cc(F)c(F)cc1F ; COc1c(F)cc(F)cc1C(C)Nc1ccn2ncc(C3=NC(C)(C)CN3)c2n1]

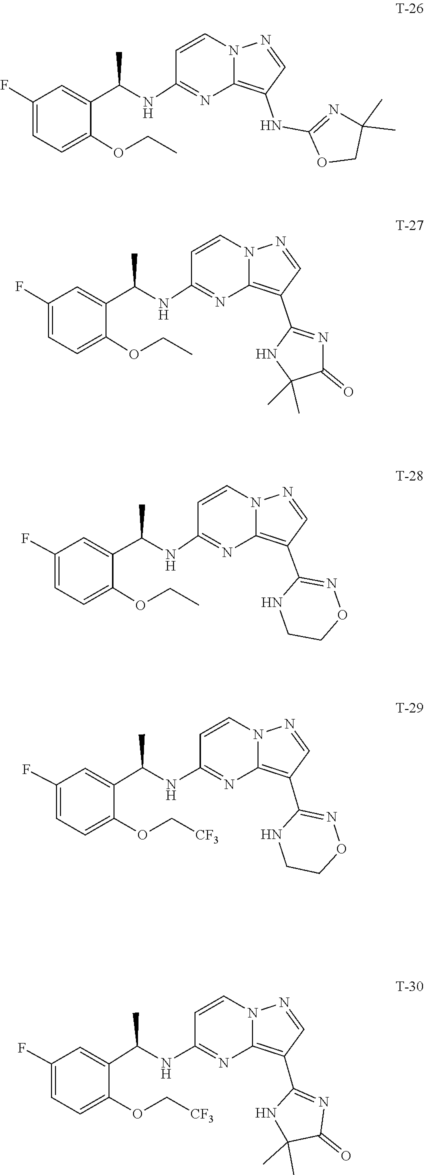 [SMILES: CCOc1ccc(F)cc1[C@@H](C)Nc1ccn2ncc(NC3=NC(C)(C)CO3)c2n1 ; CCOc1ccc(F)cc1[C@@H](C)Nc1ccn2ncc(C3=NC(=O)C(C)(C)N3)c2n1 ; CCOc1ccc(F)cc1[C@@H](C)Nc1ccn2ncc(C3=NOCCN3)c2n1 ; C[C@@H](Nc1ccn2ncc(C3=NOCCN3)c2n1)c1cc(F)ccc1OCC(F)(F)F ; C[C@@H](Nc1ccn2ncc(C3=NC(=O)C(C)(C)N3)c2n1)c1cc(F)ccc1OCC(F)(F)F]

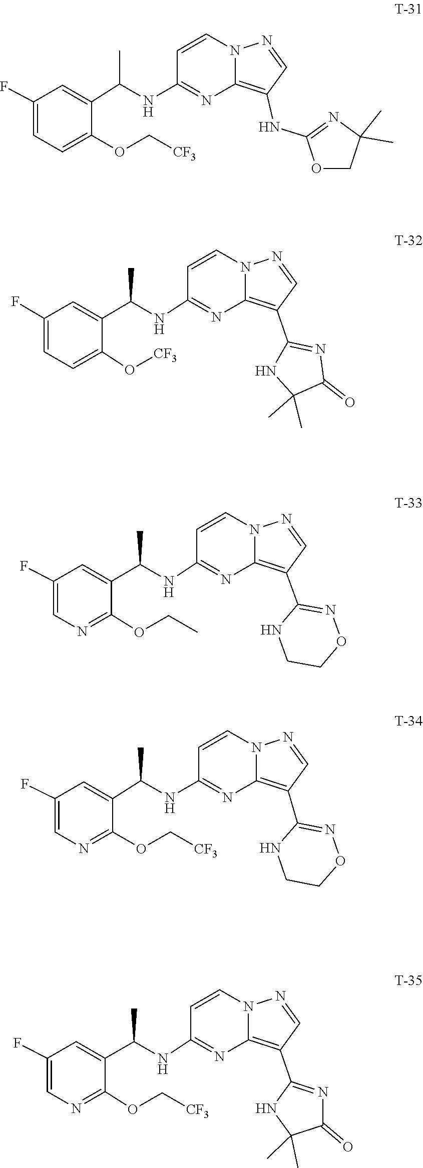 [SMILES: CC(Nc1ccn2ncc(NC3=NC(C)(C)CO3)c2n1)c1cc(F)ccc1OCC(F)(F)F ; C[C@@H](Nc1ccn2ncc(C3=NC(=O)C(C)(C)N3)c2n1)c1cc(F)ccc1OC(F)(F)F ; CCOc1ncc(F)cc1[C@@H](C)Nc1ccn2ncc(C3=NOCCN3)c2n1 ; C[C@@H](Nc1ccn2ncc(C3=NOCCN3)c2n1)c1cc(F)cnc1OCC(F)(F)F ; C[C@@H](Nc1ccn2ncc(C3=NC(=O)C(C)(C)N3)c2n1)c1cc(F)cnc1OCC(F)(F)F]

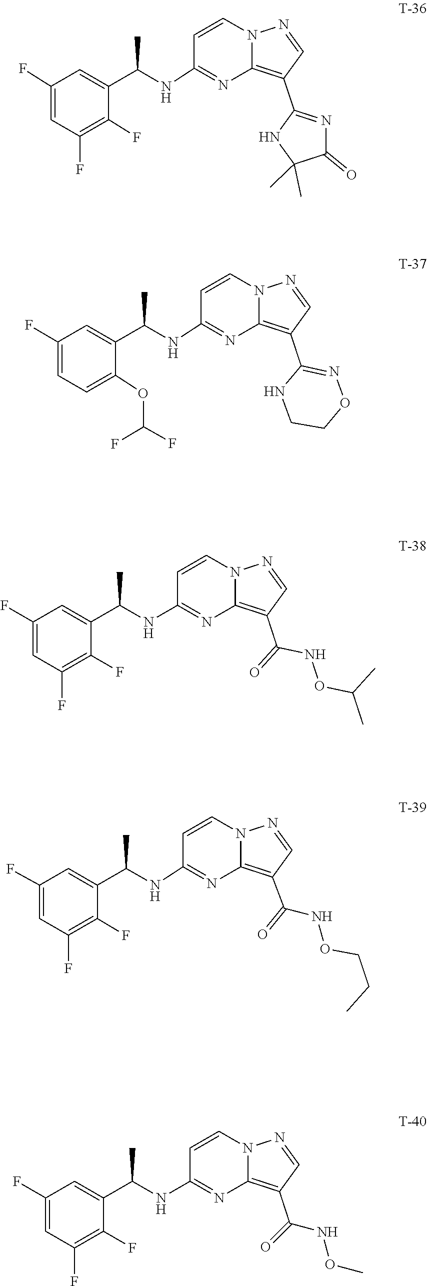 [SMILES: C[C@@H](Nc1ccn2ncc(C3=NC(=O)C(C)(C)N3)c2n1)c1cc(F)cc(F)c1F ; C[C@@H](Nc1ccn2ncc(C3=NOCCN3)c2n1)c1cc(F)ccc1OC(F)F ; CC(C)ONC(=O)c1cnn2ccc(N[C@H](C)c3cc(F)cc(F)c3F)nc12 ; CCCONC(=O)c1cnn2ccc(N[C@H](C)c3cc(F)cc(F)c3F)nc12 ; CONC(=O)c1cnn2ccc(N[C@H](C)c3cc(F)cc(F)c3F)nc12]

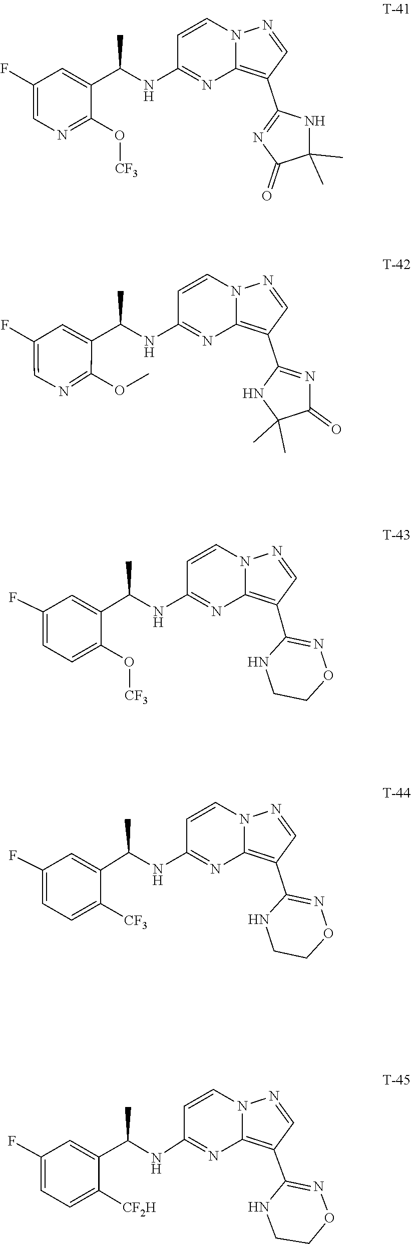 [SMILES: C[C@@H](Nc1ccn2ncc(C3=NC(=O)C(C)(C)N3)c2n1)c1cc(F)cnc1OC(F)(F)F ; COc1ncc(F)cc1[C@@H](C)Nc1ccn2ncc(C3=NC(=O)C(C)(C)N3)c2n1 ; C[C@@H](Nc1ccn2ncc(C3=NOCCN3)c2n1)c1cc(F)ccc1OC(F)(F)F ; C[C@@H](Nc1ccn2ncc(C3=NOCCN3)c2n1)c1cc(F)ccc1C(F)(F)F ; C[C@@H](Nc1ccn2ncc(C3=NOCCN3)c2n1)c1cc(F)ccc1C(F)F]

12. A pharmaceutical composition comprising i) a therapeutically effective amount of the compound of formula I, a stereoisomer, a tautomer, a crystalline form, a pharmaceutically acceptable salt, a hydrate, a solvate or a prodrug thereof of claim 1; and ii) one or more pharmaceutically acceptable carriers, diluents or excipients.

13. A method for preventing and/or treating the diseases related to pathological characteristics mediated by ROS1, NTRK, and ALK comprising administrating a therapeutically effective amount of the compound of formula I, or a stereoisomer, a tautomer, a crystalline form, a pharmaceutically acceptable salt, a hydrate, a solvate or a prodrug thereof of claim 1.

14. The method of claim 13, wherein the diseases related to pathological characteristics mediated by ROS1, NTRK, and ALK include cancer, sarcoma and pain.

15. The method of claim 14, wherein the cancer is selected from the group consisting of breast cancer, cervical cancer, colon cancer, lung cancer, stomach cancer, rectal cancer, pancreatic cancer, brain cancer, skin cancer, oral cancer, prostate cancer, bone cancer, kidney cancer, ovarian cancer, bladder cancer, liver cancer, fallopian tumor, peritoneal tumor, melanoma, glioma, glioblastoma, head and neck cancer, mastoid nephroma, leukemia, lymphoma, myeloma and thyroid tumor.
